(19) United States
(12) Patent Application Publication
(10) Pub. No.: US 2002/0052568 A1
(43) Pub. Date: May 2, 2002

(54) Joint braces and traction devices incorporating superelastic supports

(76) Inventors: Russell A. Houser, Livermore, CA (US); James G. Whayne, Chapel Hill, NC (US)

Correspondence Address: RUSSELL A. HOUSER, 1787 VERDITE STREET, LIVERMORE, CA, 94550, US

(21) Appl. No.: 09/965,542

(22) Filed: Sep. 27, 2001

Related U.S. Application Data
(60) Provisional application No. 60/236,923, filed on Sep. 28, 2000.
(60) Provisional application No. 60/272,110, filed on Feb. 28, 2001.
(60) Provisional application No. 60/295,839, filed on Jun. 6, 2001.

(51) Int. Cl. A61F 5/00

(52) U.S. Cl. 602/26; 602/23

(57) ABSTRACT

Described are anatomic braces, traction devices, and scaffolds containing superelastic supports capable of reinforcing anatomic joints or other structures, applying a directional force in response to a deflection, and/or producing a spring force to apply decompression or compression to the anatomy. The braces are intended to reinforce the back, knees, ankles, elbows, wrists, shoulders, neck, hips, or other anatomy commonly associated with a degree of twisting, rotation, bending, or other desired motion. The anatomic braces and traction devices also provide motion assistance and/or intensify the force during motion of anatomic structures. In particular, the anatomic braces and traction devices aid in standing, bending, throwing, kicking, jumping, running, walking, hitting, shooting, or other strenuous activity by inducing a directional force upon deflection. The braces alternatively apply a specific resistance at the anatomic joint to strengthen the muscles during training or rehabilitation. The braces also immobilize or stabilize joints, bones, or other anatomic structures during healing of an injury.

### CROSS REFERENCE TO RELATED APPLICATIONS

This application is related to the following applications: co-pending Provisional application Serial No. 60/236,923 entitled “Anatomic Braces with Superelastic Supports”; co-pending Provisional application Serial No. 60/272,110 entitled “Joint Braces and Scaffolds with Superelastic Supports”; co-pending Provisional application Serial No. 60/295,839 entitled “Traction Devices Incorporating Superelastic Supports”; each of which is incorporated herein by reference.

### FIELD OF THE INVENTIONS

This invention relates to devices for reinforcing and enhancing the performance of anatomic structures. More particularly, the invention relates to supports that are incorporated in braces to prevent excessive or unwanted twisting, bending, or other movement capable of causing injury or excessive stress to the anatomical joint or structures. As such, the muscles, tendons, bone interconnections, and other anatomy that enable movement at the joints are reinforced so they are less susceptible to being exposed to excess tension, stress, strain, or torque. The superelastic braces of the invention also preserve the flexibility at the joint and/or intensify the force exerted during movement of anatomic structures about the joint. In particular, the superelastic supports of the braces enhance throwing, kicking, jumping, running, walking, hitting, shooting, or other strenuous activity by providing a directional force in response to an opposing deflection.

The braces of the invention are intended to reinforce and/or strengthen the knees, ankles, elbows, wrists, shoulders (especially the rotator cuffs), back, neck, hips, or other anatomy commonly associated with a degree of twisting, rotation, bending, or other desired motion. The braces of the invention also enable applying a specific force against the muscles at the joint to strengthen the muscles during training or rehabilitation. The braces also enable applying a specific force against an injured bone, joint, or other anatomy to prevent movement during treatment of the injury. Different braces having different force characteristics and/or enabling different degrees of movement may be used during the healing process to gradually permit increased motion and accelerate the strengthening of tissue adjacent the injured bone or joint which would otherwise be immobilized.

The invention also relates to supports that are incorporated in traction devices capable of relieving stress on anatomic structures by exerting a decompression force to counteract gravitational forces, or other compressive forces, on the anatomy. The traction devices further prevent excessive or unwanted twisting, bending, or other movement capable of causing injury. The superelastic traction devices of the invention also preserve the flexibility at the joint, facilitate the response to motion about the joint, and/or intensify the force exerted during movement of anatomic structures about the joint. As such, the traction devices provide motion assistance to aid the wearer in performing an activity. In particular, the superelastic supports of the traction devices and braces enhance standing, bending, walking, or other strenuous activity by providing a directional force in response to an opposing deflection.

The traction devices of the invention are intended to apply a decompression force against, reinforce, and/or strengthen the back. Traction devices having adjustable force characteristics and/or degrees of motion may be used during the healing process to vary the amount of decompression and gradually permit increased movement to accelerate the healing and strengthening of tissue adjacent the injured bone or joint which would otherwise be immobilized.

The supports may also be incorporated into guards or shields for the head, rib, finger, pelvis, clavicle, shin, thigh, forearm, lower back, upper back, hip, foot, neck, upper arm, or other anatomy so as to distribute and/or redirect an external stress applied to a localized point or region over a larger surface area. This prevents extremely concentrated forces, which potentially cause fracture or other injury to bones, detachment of tendons, or tearing or other injury to muscles.

### DESCRIPTION OF THE RELATED ART

Current techniques for providing braces involve using an elastic or semi rigid polymer that is positioned around the joint and holds the anatomy in place. These current braces interfere with movement of the anatomy about the joint adversely impacting the normal degree of rotation, the normal amount of bending, and the force exerted upon movement of the anatomy about the joint. In addition, these current braces are limited in their ability to prevent excess rotation, bending or other motion unless they are fabricated extremely thick; however, when fabricated thick they further hinder the desired movement of the anatomy about the joint.

Another conventional brace configuration incorporates stainless steel, or other solid metal or alloy bar attached to the brace and incorporating a hinge to enable movement of the bar about the joint. These current braces are typically relatively heavy, and severely limit any motion of the anatomy thus adversely impact the performance of the anatomic structures. As such they greatly inhibit the normal rotation, bending, or other motion that inherently produces an applied force and elicits a desired response (e.g. walking, running, hitting, throwing, or other activity).

Current techniques for providing traction devices involve pneumatic bladders that may be adjusted to vary the amount of decompression force applied to the back or other joint. These conventional traction devices also interfere with movement of the anatomy about the joint adversely impacting the normal degree of rotation, bending, and force exerted upon movement of the anatomy about the joint.

A need thus exists for braces that incorporate supports capable of being deflected a predetermined amount in response to an external force and exert an opposing force in response to the deflection. As such these braces preserve or enhance the natural motion of the anatomy about the joint and prevent excess twisting, bending, or other motion capable of resulting in injury.

A need also exists for traction devices that incorporate superelastic supports capable of being deflected a predetermined amount in response to an external force and exert an opposing force in response to the deflection. As such these traction devices provide decompression to anatomic structures, and prevent excess twisting, bending, or other motion capable of resulting in injury.

In addition there is a need for braces and traction devices that provide a predetermined resistance to motion so as to immobilize, stabilize, or strengthen anatomic structures during rehabilitation or recuperation from an injury, or training.

### SUMMARY OF THE INVENTION

The embodiments of the present invention provide braces and traction devices that contain superelastic supports that elastically return towards their baseline, or annealed configuration when deflected by an external force. As such these superelastic supports may be utilized in braces to produce an opposing force once deflected and enhance walking, running, standing, bending, jumping, throwing, rotating, hitting, shooting, swinging, or other motion associated with physical activity. The supports also correct anatomic abnormalities or athletic form for a specific activity by directing the anatomy of the joint into the desired position during rest, walking, running, throwing, swinging, or otherwise exercising.

The embodiments of the present invention also provide scaffolds and braces that contain superelastic supports to reinforce, guard, or shield the head, fingers, ribs, forearm, upper arm, shin, thigh, foot, hip, lower back, upper back, clavicle, other bone, or other joint by preventing localized stressing of the anatomy that may cause fracture, tearing, or other injury. The embodiments also provide scaffolds and braces incorporating superelastic supports that reinforce, apply continuous compressive force against, and/or stabilize anatomic structures thereby treating sprains, fractures, torn muscles, torn tendons, or other injury. In addition, the embodiments provide scaffolds and braces that reinforce or apply continuous force on the sternum, ribs, or other anatomic region and ensure rapid and evenly distributed healing of the sternum, ribs, or other anatomic region after open-heart surgery involving a median sternotomy, thoracotomy, or thoracostomy, or other invasive procedure.

The embodiments of the present invention also provide traction devices that provide decompression against specific anatomic structures such as the back, and/or produce an opposing force upon deflection. The embodiments of the present invention provide traction devices that contain superelastic supports to provide decompression against specific anatomic structures, reinforce the anatomic structures, apply localized continuous compressive force against specific anatomic structures, and/or stabilize anatomic structures. As such, the traction devices aid in healing by reinforcing specific regions and relieving stress exerted upon other regions of the body during rest, standing, bending, walking, running, or other strenuous activity.

The above described and many further features and advantages of the present invention will be elaborated in the following detailed description and accompanying drawings.

### BRIEF DESCRIPTION OF DRAWINGS

FIGS. 1a and b show perspective views of knee that are reinforced with two brace configurations, each containing superelastic supports;

FIGS. 2a and b show a side view and a front view of a knee that is reinforced with a brace fabricated from superelastic wire supports;

FIGS. 3a and b show a side view and a front view of a knee that is reinforced with an alternative brace embodiment fabricated from superelastic wire supports;

FIGS. 4a and b show a side view and a front view of a knee that is reinforced with an alternative brace embodiment fabricated from superelastic wire supports;

FIGS. 5a to f show sequential steps of fabricating a knee brace fabricated from superelastic wire supports;

FIG. 5g shows a cross-sectional view of a clamp used to stress the superelastic wire supports into the desired 3-dimensional geometry while thermally forming the brace;

FIGS. 6a and b show a side view and a front view of a knee that is reinforced with a brace fabricated from a superelastic wire support stressed and thermally formed into the desired shape as shown in FIGS. 5a to f;

FIGS. 7a and b show end views of the two pieces that, when bonded, define an interconnect embodiment used to secure intersecting sections of at least one superelastic wire support;

FIGS. 8a and b show end views of the two pieces that, when bonded, define an alternative interconnect embodiment used to secure intersecting sections of at least one superelastic wire support;

FIG. 9 shows an end view of a hinge stop embodiment used to limit the movement of a superelastic wire support;

FIGS. 10a and b show a side view and a front view of a knee that is reinforced with an alternative brace embodiment fabricated from at least one superelastic wire support;

FIGS. 11a and b show a side view and a front view of a knee that is reinforced with an alternative brace embodiment fabricated from at least one superelastic wire support;

FIGS. 12a and b show a side view and a front view of a knee that is reinforced with an alternative brace embodiment fabricated from at least one superelastic wire support;

FIGS. 13a and b show a side view and a front view of a knee that is reinforced with a brace fabricated from superelastic wire supports;

FIGS. 14a to d show a perspective view, a side-sectional view and two front views of a stop embodiment for use with a knee that is reinforced with superelastic wire supports;

FIGS. 15a and b show a side view and a front view of a knee that is reinforced with an alternative brace embodiment fabricated from superelastic wire supports;

FIGS. 16a to c show perspective views of ankles that are reinforced with two brace configurations, each containing superelastic supports;

FIG. 17 shows a perspective view of an elbow that is reinforced with a brace containing superelastic supports;

FIGS. 18a to c show perspective views of wrists that are reinforced with three brace configurations, each containing superelastic supports;

FIGS. 19a and b show perspective views of shoulders that are reinforced with two brace configurations, each containing superelastic supports;

FIGS. 20a to c show perspective views of backs that are reinforced with three brace configurations, each containing superelastic supports;

FIGS. 21a and b show perspective views of two back brace embodiments, each fabricated from at least one superelastic wire support;

FIGS. 22a and b show rear views of an alternative back brace embodiment that incorporates removable pockets into which superelastic supports may be inserted and removed to vary the stiffness and spring force response;

FIG. 22c shows a rear view of an alternative back brace embodiment that incorporates superelastic wire supports;

FIGS. 22d to i show cross-sectional views of various back brace configurations taken along line A-A for the embodiments in FIGS. 22a to c;

FIG. 23a shows a rear view of an alternative back brace embodiment that consists of a jacket encompassing at least one superelastic wire support;

FIG. 23b shows a perspective view of an alternative back brace embodiment that consists of at least one superelastic wire support fabricated as a jacket;

FIGS. 23c to e show an end-view taken along line B-B of the superelastic wire support(s) in FIG. 23b.

FIGS. 24a and b show an end view and a flattened view of a back brace fabricated from at least one superelastic wire support designed to provide traction to the spine and other structures of the back;

FIGS. 25a and b show an end view and a flattened view of an alternative back brace embodiment that incorporates superelastic wire supports to provide traction;

FIGS. 26a and b show an end view and a flattened view of an alternative back brace embodiment that incorporates superelastic wire supports to provide traction and provides a mechanism to increase and decrease the amount of traction exerted by the back brace;

FIGS. 27a and b show an end view and a flattened view of an alternative back brace embodiment that utilizes at least one superelastic wire support to provide traction and incorporates at least one mechanism to vary the degree of traction;

FIGS. 28a and b show an end view and a side view of an alternative back brace embodiment that incorporates at least one superelastic support fabricated to exert traction against anatomic structures of the back;

FIGS. 29a and b show perspective views of necks that are reinforced with two brace configurations, each containing superelastic supports;

FIG. 30 shows a perspective view of a neck restraint fabricated from at least one superelastic wire support and secured to a helmet or other head protective device;

FIG. 31 shows a perspective view of an alternative neck restraint embodiment fabricated from at least one superelastic wire support and secured to a helmet or other head protective device;

FIG. 32a shows a side view of a head restraint designed to limit abrupt movement of the head about the neck, thereby reducing stress on the neck during an accident or other dramatic change in motion;

FIGS. 32b and c show a side view and a top view of an alternative head restraint embodiment;

FIG. 32d shows a side view of a removable restraining pin used with the head restraint embodiment in FIGS. 32b and c;

FIGS. 32e and f show a top view and a side view of an alternative head restraint embodiment;

FIG. 33 shows a side view of a helmut that contains superelastic supports;

FIGS. 34a to c show side views of implantable or external bone scaffolds used to reinforce bone fractures or promote bone enlargement;

FIGS. 35a to c show side views of implantable or external bone joint scaffolds used to reinforce adjacent bones at anatomic joints;

FIG. 36 shows a side view of an alternative implantable or external bone scaffold embodiment used to reinforce bone fractures or promote bone enlargement;

FIGS. 37a and b show side views of two implantable or external bone joint scaffold embodiments used to reinforce adjacent bones at anatomic joints;

### DETAILED DESCRIPTION OF PREFERRED EMBODIMENTS

The following is a detailed description of the presently best-known modes of carrying out the inventions. This detailed description is not to be taken in a limiting sense, but is made merely for the purpose of illustrating the general principles of the inventions.

This specification discloses a number of embodiments, mainly in the context of reinforcement, abnormality correction, and performance enhancement for traction devices, joint braces, and anatomic scaffolds. Nevertheless, it should be appreciated that the structures are applicable for use in other indications involving devices that are used to provide decompression against anatomic structures, exert continuous force against anatomic structures once positioned, restrict motion to a desired track, and/or exert a desired force in response to an externally induced deflection. The embodiments of the invention are configured for the human anatomy; however, it should be noted that the embodiments of the invention might be tailored to other species such as horses by changing the geometry and sizes of the structures.

The embodiments of the invention provide supports fabricated from superelastic shape memory alloys. These superelastic supports elastically deform upon exposure to an external force and return towards their preformed shape upon reduction or removal of the external force. The superelastic support members may exhibit stress-induced martensite characteristics in that they transform from the preshaped austenite form to the more soft and ductile martensite form upon application of stress and transform back toward the more strong and hard austenite form once the stress is released or reduced; this depends on the composition of the superelastic shape memory alloys which affects the temperature transition profile. Superelastic shape memory alloys also enable straining the material numerous times without plastically deforming the material. Superelastic shape memory alloys are light in weight, and exhibit excellent tensile strengths such that they may be used as traction devices, joint braces, anatomic scaffolds, guards, shields, or other devices without dramatically increasing the weight of the device, or making the device thick or bulky. The utility of superelastic materials in supports for traction devices and joint braces is highlighted by the inherent properties of such materials; they are able to withstand continuous and frequent deflections without plastically deforming or observing fatigue failures.

These supports may also be elastically deflected into small radii of curvatures and return towards their preformed configuration once the external force causing the deflection is removed or reduced. Many other known metal, alloy, and thermoplastic materials plastically deform or break when deflected into similar radii of curvature or exposed to comparable strains; as such these other metal, alloy, and thermoplastic materials do not return towards their original configuration when exposed to the amount of deflection such supports are expected to endure. Therefore superelastic supports may inherently incorporate flex regions, which conventional traction devices and joint braces are unable to accommodate, thereby eliminating the need for two or more components being connected through a hinge structure that requires pivot points between the two or more components.

In addition, superelastic supports are able to apply decompression or compression forces by taking advantage of the spring characteristics of such materials thereby eliminating the need for pneumatic mechanisms designed to alter the rigidity of fluid bladders by changing the pressures inside the bladders thus vary the forces (decompression or compression) exerted by the traction devices or joint braces. Thus the complexity and cost of traction devices and joint braces that incorporate superelastic supports is significantly reduced when compared to conventional traction devices and joint braces. In addition, superelastic supports permit deflections into smaller radii of curvature than other metals, alloys, and polymers resulting in larger strains, and they are capable of exerting substantial force when deflected, ensuring the superelastic supports return towards their preformed shape after being elastically deformed.

Superelastic supports are preferably fabricated from shape memory alloys (e.g. nickel titanium) demonstrating stress-induced martensite at ambient temperature. Of course, other shape memory alloys may be used and the superelastic material may alternatively exhibit austenite properties at ambient temperature. The composition of the shape memory alloy is preferably chosen to produce the finish and start martensite transformation temperatures (Mf and Ms) and the start and finish austenite transformation temperatures (As and Af) depending on the desired material response. When fabricating shape memory alloys that exhibit stress induced martensite the material composition is chosen such that the maximum temperature that the material exhibits stress-induced martensite properties (Md) is greater than Af and the range of temperatures between Af and Md covers the range of ambient temperatures the support members are exposed. When fabricating shape memory alloys that exhibit austenite properties and do not transform to martensite in response to stress, the material composition is chosen such that both Af and Md are less than the range of temperatures the supports are exposed. Of course, Af and Md may be chosen at any temperatures provided the shape memory alloy exhibits superelastic properties throughout the temperature range they are exposed. Nickel titanium having an atomic ratio of 51.2% Ni and 48.8% Ti exhibits an Af of approximately −20° C.; nickel titanium having an atomic ratio of 50% Ni to 50% Ti exhibits an Af of approximately 100° C. [Melzer A, Pelton A. Superelastic Shape-Memory Technology of Nitinol in Medicine. Min Invas Ther & Allied Technol. 2000: 9(2) 59-60].

Such superelastic materials are able to withstand strain as high as 10% without plastically deforming. As such, these superelastic materials are capable of elastically exerting a force upon deflection. Materials other than superelastic shape memory alloys may be used as supports provided they can be elastically deformed within the temperature, stress, and strain parameters required to maximize the elastic restoring force thereby enabling the brace to exert a directional force in response to an induced deflection. Such materials include other shape memory alloys, bulk metallic glasses, amorphous Beryllium, suitable ceramic compositions, spring stainless steel 17-7, Elgiloy™, superelastic polymers, etc.

The embodiments of the invention provide braces for any anatomic joint. In particular, the braces of the invention contain superelastic supports that are capable of preventing excess twisting, abnormal rotation, unwanted bending or other deleterious motion capable of causing injury to the muscles, tendons, bones, or other anatomic structures around the joint. The superelastic braces also exert a directional force in response to an opposing deflection. As such, these superelastic supports enhance activities such as walking, running, hitting, throwing, shooting, swinging, kicking, jumping, or other physical motion by utilizing the elastic recoil of the supports to augment the natural force exerted by the muscles. The embodiments of the invention also provide superelastic supports capable of exerting a desired force profile throughout the anatomic region to correct abnormalities which may be corrected through continuous urging towards the desired orientation, and/or treat fractures, sprains, or other injury requiring some degree of immobilization or stabilization. The embodiments of the invention also enable correcting incorrect or sub optimal athletic forms by urging the joint to move in a predetermined path. The stiffness of the superelastic supports may be selected and distributed to produce a predefined stiffness profile throughout the brace and/or exert varying amounts of force throughout the surface of the brace.

The embodiments of the invention provide knee braces, ankle braces, elbow braces, wrist braces, shoulder supports, hip braces, back braces, and neck braces which incorporate superelastic supports to reinforce the joints and prevent abrupt twisting, bending, or separation of the joints, which may result in injury.

The embodiments of the invention provide traction devices for any joint or anatomic structure benefiting from reinforcement and/or decompression. In particular, the traction devices of the invention contain superelastic supports that exert decompression forces against anatomic structures to reduce stresses on the anatomy; this accelerates healing and/or prevents injury to the anatomy. In addition, the traction devices are capable of preventing excess twisting, abnormal rotation, unwanted bending or other deleterious motion capable of causing injury to the muscles, tendons, bones, or other anatomic structures around the joint. The superelastic supports also exert a directional force in response to an opposing deflection. As such, these superelastic supports provide traction against anatomic structures to decrease stresses placed on the anatomy by urging the anatomic structures to separate while maintaining the ability to perform daily activities such as standing, walking, running, hitting, throwing, shooting, swinging, kicking, jumping, or other physical motion by utilizing the elastic recoil of the supports to augment the natural force exerted by the muscles.

The embodiments of the invention provide traction devices for the back, knee braces, ankle braces, elbow braces, wrist braces, shoulder supports, hip braces, and neck braces that incorporate superelastic supports to reinforce joints, and prevent abrupt twisting, bending, or separation of anatomic structures, which may hinder healing, result in injury, or produce tensile-related or torsional-related pain. The traction devices of the invention also provide decompression to anatomic structures to decrease the stress and strain on muscles and underlying anatomy; this aids in healing and decreases compression-related pain accompanying daily activities for patients inflicted with back abnormalities. The traction devices for the back address pre-op and post-op patient needs by providing the flexibility to change the stiffness and the amount of decompression (or compression) required during the specific phase of treatment.

For all traction device and brace embodiments containing superelastic supports described below, the supports may be embedded in a flexible covering (not shown in all drawings). The covering may only cover individual supports or may encompass all or a subset of superelastic supports between covering layers. The covering may be fabricated from neoprene, fabric mesh, LYCRA™, SPANDEX™, leather, chamois, silicone, polyurethane, rubber, PEBAX™, nylon, polyester, other cushioning material typically used in braces and demonstrating excellent elasticity, or a combination of these materials. Since the superelastic supports provide the decompression and/or reinforcing structure, the covering may be fabricated extremely thin. This further ensures the brace is capable of maintaining or enhancing the motion of the joint and does not hinder movement of the anatomy about the joint.

The covering may be attached to the superelastic supports by dipping the supports, laminating layers around the supports, adhesively bonding layers together and/or to the supports, ultrasonic welding, thermal bonding, radio frequency welding, laser welding the layers, sewing, other manufacturing process capable of encompassing the superelastic supports between layers of covering, or a combination of these bonding processes. The covering layers may be attached to each other and/or the supports when embedding the supports within layers of covering material. The covering layers may be fabricated with perforations to wick away sweat, provide pathways for air to pass, or other purpose.

The superelastic supports 60 of the traction devices and joint braces are fabricated in the desired pattern of links (e.g. tightening links, spring links, and links having other purposes and characteristics) to tailor the desired spring characteristics, radial stiffness, and axial stiffness to optimize the traction device or brace to the desired decompression of the anatomy, and/or motion of the joint. The ability to change parameters of the various links may be accomplished by the inherent properties of the annealed superelastic supports, or other components may be used to change the geometry, attachment points, lengths, other variable, or a combination of variables that affect the links' spring characteristics. The superelastic supports may contain any number of tightening links, spring links, and links having other purposes and characteristics.

The superelastic supports may be fabricated from at least one rod, wire, band, bar, tube, sheet, ribbon, other raw material having the desired pattern, cross-sectional profile, and dimensions, or a combination of cross-sections. The superelastic supports are cut into the desired pattern and are thermally formed into the desired 3-dimensional geometry. Alternatively the superelastic supports may be fabricated as a plane for their preformed orientation, and secured as a 3-dimensional geometry around the joint by tying opposite ends with knots, applying Velcro or other attachment means between opposite ends, or using other removable securing process. The rod, wire, band, bar, sheet, tube, ribbon, or other raw material may be fabricated by extruding, press-forging, rotary forging, bar rolling, sheet rolling, cold drawing, cold rolling, using multiple cold-working and annealing steps, casting, or otherwise forming into the desired shape. Then the supports must be cut into the desired length and/or pattern. Conventional abrasive sawing, water jet cutting, laser cutting, EDM machining, photochemical etching, or other etching techniques may be employed to cut the supports from the raw material.

Ends or any sections of the rod, wire, band, sheet, tubing, ribbon, or other raw material may be attached by laser welding, adhesively bonding, soldering, spot welding, or other attachment means. This encloses the superelastic supports to provide additional reinforcement, eliminate edges, or other purpose. Multiple rods, wires, bands, sheets, tubing, ribbons, other raw materials, or a combination of these may be bonded to produce a composite superelastic support and form the skeleton of the brace.

For several of the traction device and brace embodiments below, the superelastic supports are fabricated from a wire, rod, band or bar of nickel titanium material cut to the desired length and thermally formed into the desired 3-dimensional configuration. Alternatively, rod, wire, band, bar, sheet, tube, ribbon, combination, or other geometry of superelastic supports may be used. When thermally forming superelastic supports, the superelastic material(s), previously cut into the desired pattern and/or length, are stressed into the desired resting configuration over a mandrel or other forming fixture having the desired resting shape of the brace, and the material is heated to between 300 and 600 degrees Celsius for a period of time, typically between 15 seconds and 10 minutes. Once the volume of superelastic material reaches the desired temperature, the superelastic material is quenched by inserting into chilled water or other fluid, or otherwise allowed to return to ambient temperature. As such the superelastic supports are fabricated into their resting configuration. When extremely small radii of curvature are desired, multiple thermal forming steps may be utilized to sequentially bend the rod, wire, band, sheet, tubing, ribbon or other raw material into smaller radii of curvature.

When fabricating the superelastic supports from tubing, the raw material may have an oval, circular, rectangular, square, trapezoidal, or other cross-sectional geometry capable of being cut into the desired pattern. After cutting the desired pattern of tightening links and support links, the supports are formed into the desired shape, heated, for example, between 300° C. and 600° C., and allowed to cool in the preformed geometry to set the shape of the support members.

When fabricating the supports from flat sheets of raw material, the raw material may be configured with at least one width, W, and at least one wall thickness, T, throughout the raw material. As such, the raw sheet material may have a consistent wall thickness, a tapered thickness, or sections of varying thickness. The raw material is then cut into the desired pattern of tightening links and spring links, and thermally shaped into the desired 3-dimensional geometry. Opposite ends of the thermally formed support member may be secured by using rivets, shrink tubing, applying adhesives, welding, soldering, mechanically engaging, utilizing another bonding means, or a combination of these bonding methods. Opposite ends of the thermally formed supports may alternatively be free-floating to permit increased flexibility.

Once the supports are fabricated and formed into the desired 3-dimensional geometry, the supports may be electropolished, tumbled, sand blasted, chemically etched, ground, or otherwise treated to remove any edges and/or produce a smooth surface.

Holes, slots, notches, other cut-away areas, or regions of ground material may be incorporated in the support design to tailor the stiffness profile of the support. Such holes, slots, notches, or other cut-away areas are also beneficial to increasing the bond strength or reliability when attaching the covering(s) to the superelastic supports. Cutting and treating processes described above may be used to fabricate the slots, holes, notches, cut-away regions, and/or ground regions in the desired pattern to taper the stiffness along the support, focus the stiffness of the supports at the tightening links, reinforce the spring links of the support, or otherwise customize the stiffness profile of the brace.

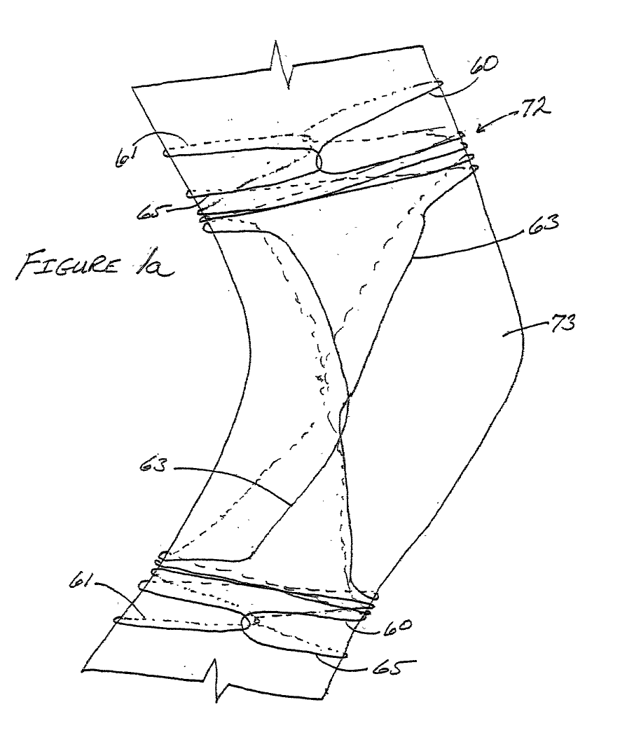

FIGS. 1a and b show two knees, each reinforced by a knee brace 72 embodiment. The knee braces 72 incorporate superelastic supports 60 distributed throughout the braces. The distribution and the characteristics of the superelastic support(s) determine the purpose for the brace and the amount of force the brace is capable of exerting in response to an external force such as a deflection. The superelastic supports have at least one width, W, at least one length, and at least one thickness, T, configured to produce the desired stiffness and force profile. The width, length, and/or thickness may vary throughout the superelastic supports to vary the stiffness profile and resulting response to movement.

The knee braces in FIGS. 1a and b incorporate two tightening links 65 connected with at least one spring link 63. The superelastic supports 60 are shown as solid lines on one side of the leg and dotted lines 61 on the opposite side of the leg. During manufacture the cross-section of each superelastic support link may be a circular rod, a rectangular band, a circular or elliptical wire, a square ribbon, a donut shaped tube, or other geometry that provides the desired stiffness to impart the reinforcing and spring forces. It should be noted that the orientation of the superelastic support links relative to the leg depends on the purpose for the brace and helps dictate the restriction of abnormal motion and the spring characteristic of the brace. The embodiment in FIG. 1a has a total of four spring links 63, two on each side of the leg; the spring links 63 extend from the tightening links 65 above the knee 73 to the tightening links 65 below the knee. The spring links 63 permit the desired amount of movement about the joint and exert a desired amount of elastic recoil in response to an induced deflection. The embodiment in FIG. 1b has two spring links 63 on one side of the leg, and six spring links 63 on the opposite side of the leg. It should be noted that any number of spring links may be chosen depending on the manufacturing process, the desired spring constant, and the desired stiffness profile.

The tightening links 65 are configured to enlarge in response to an external force to enable positioning the brace over the leg and return towards their preformed shape once positioned. The external force causing the tightening links 65 to enlarge may consist of squeezing opposing segments of the tightening links together to expand the inner diameter, spread segments of the tightening links apart, or otherwise manipulating the tightening links to produce an expanded diameter. Once the tightening links 65 are allowed to return towards their resting configuration, the tightening links 65 provide a suitable compression around the leg regions above and below the knee to stabilize the position of the spring links and ensure suitable operation of the brace. The tightening links 65 are configured to compress in diameter upon bending, twisting, or other unwanted motion further anchoring the superelastic supports and preventing excess motion, which could cause injury to the knee.

The superelastic supports of the knee braces in FIGS. 1a and b may be thermally formed at any orientation between straight and bent at the maximum deflection a knee allows. The spring links 63 are preshaped to orient the knee braces at the desired resting orientation, depending on the desired activity. When the superelastic supports are preshaped in a generally straight position, the knee brace urges the knee joint towards the resting, straight position; when the leg is deflected backwards during walking, running, kicking, or other motion, the energy causing the spring links 63 to deflect produces a spring force that produces an elastic recoil of the spring links once the deflection force is reduced or removed, consistent with a running, walking, or kicking motion. The stiffness of the spring links 63 determines the force required to deflect the spring links and the amount of elastic recoil. When the superelastic supports of the knee brace are thermally formed such that the resting orientation is the bent position, reflecting the orientation of the leg at the any predetermined point during the walking, or running motion, a stored energy is produced when the spring links 63 are deflected towards the straight orientation. Once the deflection force causing the spring links 63 to straighten is reduced or removed, the elastic recoil of the spring links 63 causes the leg to return towards the bent orientation, increasing the force exerted by the muscles, especially the hamstring, during walking or running.

The spring links 63 of the knee braces are also configured to prevent twisting or excess bending of the leg anatomy about the knee 73. The stiffness of the spring links 63, and the cross-sectional geometry determine the restriction of rotation and/or limitation of motion permitted by the knee brace 72 at the knee joint.

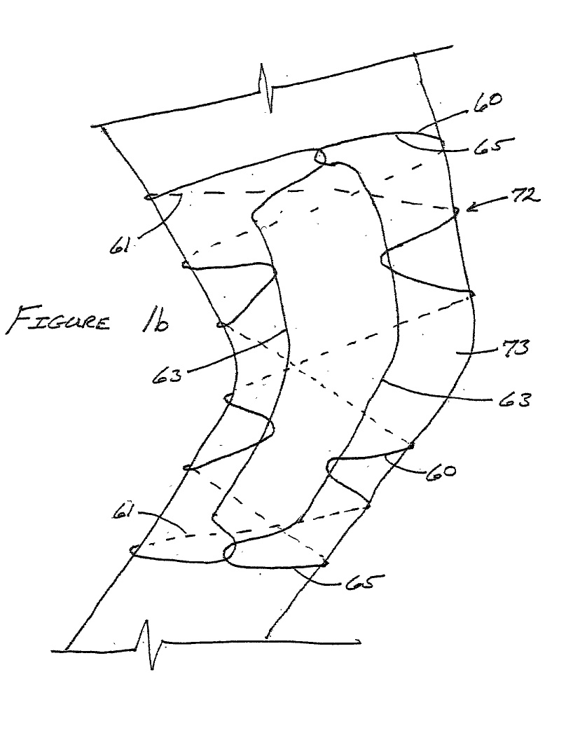

FIGS. 2a and b show a side view and a front view of a knee reinforced by a knee brace 72 fabricated from at least one superelastic support (preferably, but not limited to, a wire) 60. This knee brace 72 embodiment incorporates at least one superelastic wire support (in this case a single wire having a diameter between 0.060″ and 0.200″ is used) wrapped into the desired 3-dimensional geometry designed to support the knee and produce an integrated hinge restricting the motion of the joint to a desired tract. The distribution and the characteristics of the superelastic wire support(s) determine the purpose for the brace and the amount of force the brace is capable of exerting in response to an external force such as a deflection. The superelastic wire supports have at least one diameter, D, for circular wires (a major axis and a minor axis dimension for elliptical, oval, rectangular, trapezoidal, or other geometry wires), and at least one length configured to produce the desired stiffness and force profile. The diameter and/or length may vary throughout the superelastic wire supports to vary the stiffness profile and resulting response to movement.

The knee braces in FIGS. 2a and b incorporate two tightening links 65 connected with at least one spring link 63. The spring link 63 defines the integrated hinge, which permits flexion of the knee and determines the response of the brace to such flexion. During manufacture the cross-section of each superelastic support link may be a circular rod, a rectangular band, a circular or elliptical wire, a square ribbon, a donut shaped tube, or other geometry that provides the desired stiffness to impart the reinforcing and spring forces; in this embodiment, the superelastic support links are a circular or elliptical wire wrapped into the desired pattern. It should be noted that the orientation of the superelastic support links relative to the leg depends on the purpose for the brace and helps dictate the restriction of abnormal motion and the spring characteristic of the brace. The embodiment in FIG. 2a has two spring links 63, one on each side of the leg; the spring links 63 extend from a tightening link 65 located above the knee 73 to a tightening link 65 below the knee. The spring links 63 permit the desired amount of movement about the joint and exert a desired amount of elastic recoil in response to an induced deflection. It should be noted that any number of spring links might be chosen depending on the manufacturing process, the desired spring constant, and the desired stiffness profile.

The tightening links 65 are configured to enlarge in response to an external force to enable positioning the brace over the leg and return towards their preformed shape once positioned. The external force causing the tightening links 65 to enlarge may consist of squeezing opposing segments of the tightening links together to expand the inner diameter, spread segments of the tightening links apart, or otherwise manipulating the tightening links to produce an expanded diameter. Once the tightening links 65 are allowed to return towards their resting configuration, the tightening links 65 provide a suitable compression around the leg regions above and below the knee to stabilize the position of the spring links and ensure suitable operation of the brace. The tightening links 65 are further attached to the leg using locking bands 96 that extend around the opposite side of the leg from the tightening links 65 and may be tightened and secured with Velcro attachment mechanisms, ratchet locking mechanisms, buckling means, screw-type mechanism, or other attachment components.

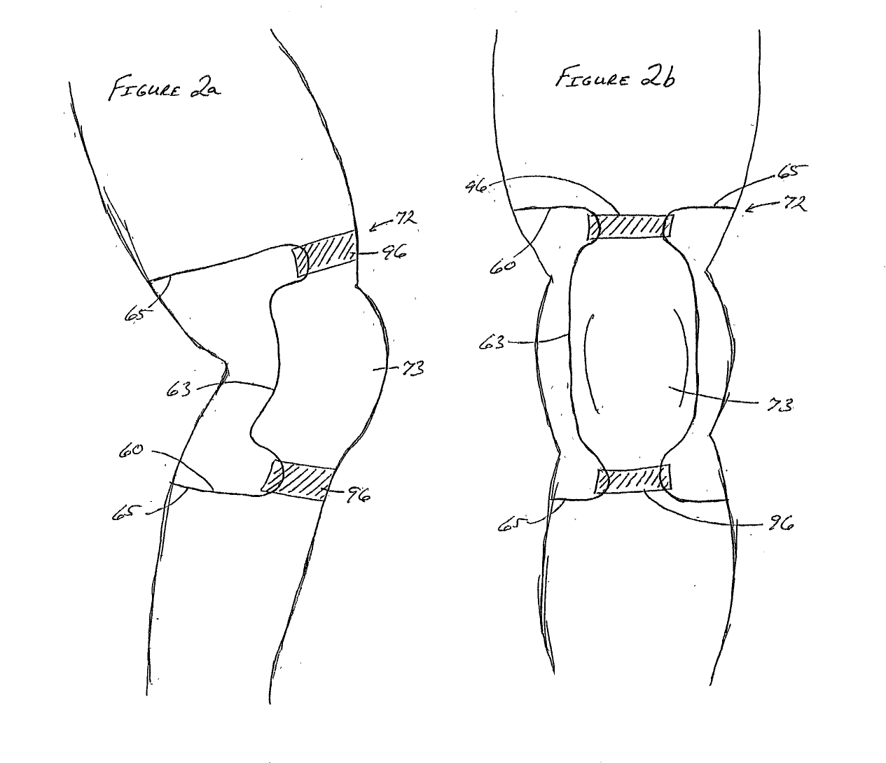

FIGS. 3a and b show a side view and a front view of an alternative knee brace embodiment that contains superelastic wire supports. As previously described, the knee brace may incorporate at least one superelastic wire support 60 embedded in at least one covering (not shown). As previously described, the covering(s) may embed individual links of the superelastic supports, a subset of the superelastic supports, or the entire superelastic support structure. The superelastic wire support structures 60 in FIGS. 3a and b contain two tightening links 65, one located along the thigh (or other location above the knee), and one located along the calf (or other location below the knee). The tightening links 65 are connected with one spring link 63 on each side of the leg. The tightening links 65 are removably attached to the leg using locking bands 96, as described above.

The superelastic wire support structures previously described for the knee braces above may additionally be modified accordingly for ankle, elbow, wrist, shoulder, back, hip, neck, other joint braces, or other anatomic scaffolds. Similarly, the superelastic wire support structures described below for knee braces, back braces, and neck restraints may modified for other anatomic joints or structures.

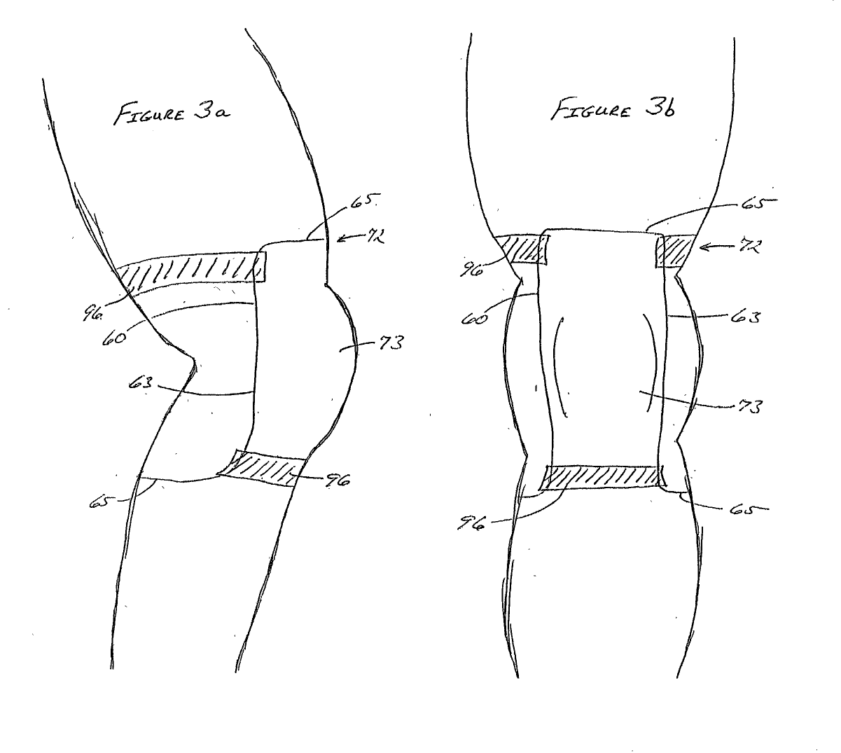

FIGS. 4a and b show a side view and a front view of a knee brace 72 that incorporates a superelastic wire support structure 60 wound into a desired pattern of tightening links 65 and spring links 63. This superelastic support structure 60 embodiment prevents over-rotating, excess twisting, unwanted bending or separation of the tissues at the knee 73. The spring links 63 for this embodiment consists of the superelastic wire support 60 wound into a spiraling coil designed to function as the integrated hinge. This integrated hinge design directs the movement of the knee brace along the spiral coil to direct movement of the knee along the desired tract. As such, the superelastic wire support structures of the knee braces enable directing motion of the leg in the correct or optimal form (depending on the activity), prevent injury of the muscles and tendons at the knee joint due to dramatic over-stressing or chronic fatiguing, and increase the applied force of the motion by providing an elastic recoil of the superelastic wire support structure due to a deflection force. The knee brace may alternatively be fabricated extremely stiff for activities where immobilizing or restricting the movement of the knee is required.

The knee brace 72 in FIGS. 4a and b incorporates a first tightening link 65 located along the thigh, a second tightening link 65 located along the calf, one spring link 63 located on each side of the arm and interconnecting the first and second tightening links. Each spring link 63 is coiled into a spiral to define the hinge, as described above. It should be noted that the two sets of spring links 63 for this embodiment permits a specific degree of rotation and bending about the knee, depending on the activity, and provides additional spring force to aid the response of the knee to a deflection force. This is essential to optimize the motion of the leg structures at the knee during activities such as walking, running, kicking, skiing, surfing, skating, snowboarding, or other endeavors associated with a degree of rotation and bending of the knee.

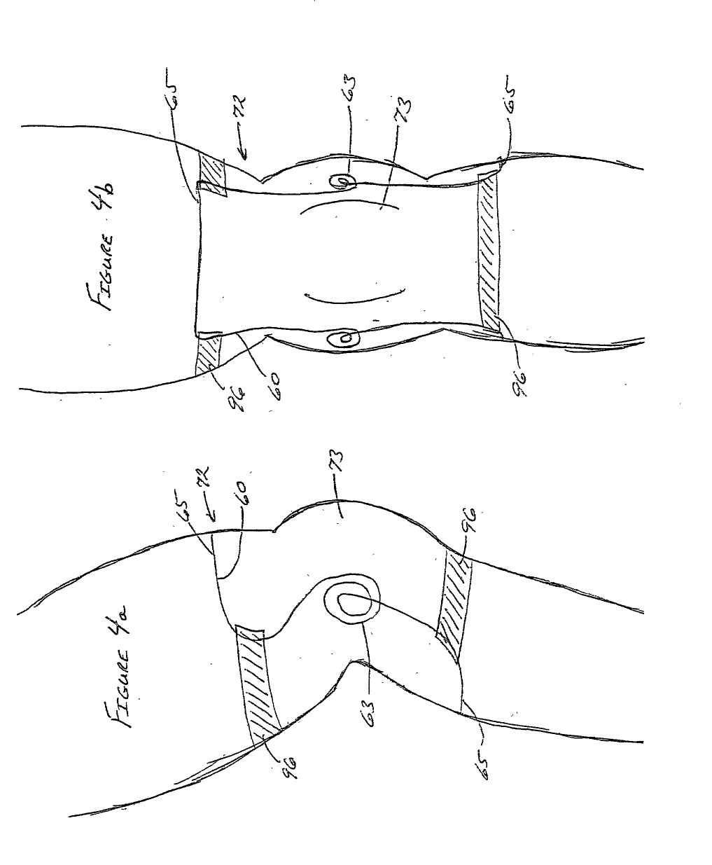

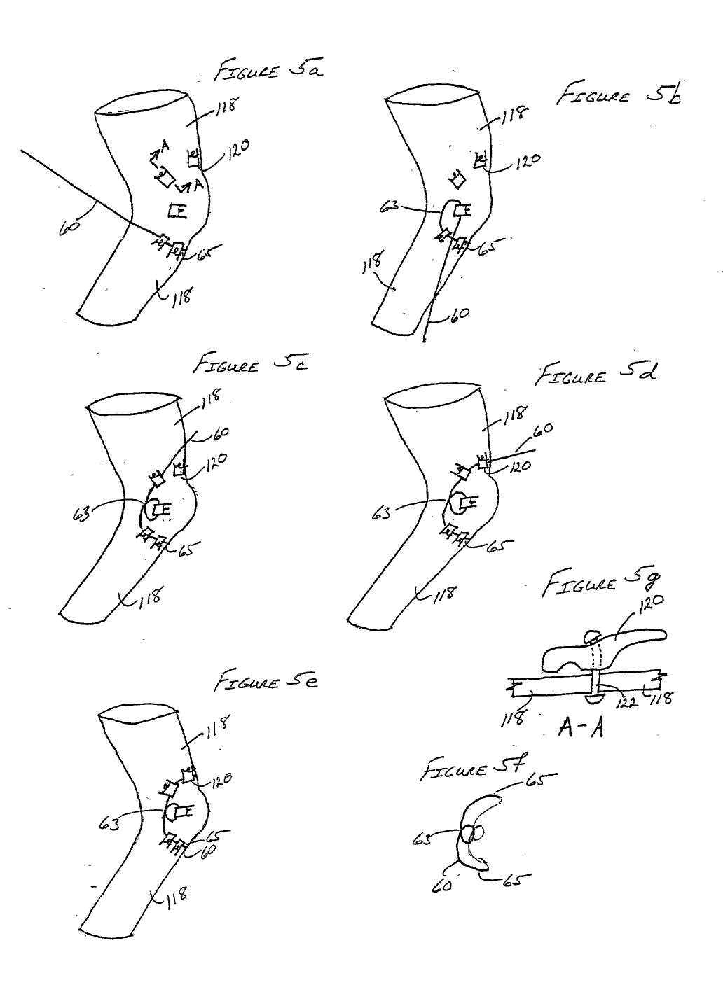

FIGS. 5a to f show sequential steps in fabricating the knee braces of the invention from superelastic wire supports. Thermal forming fixtures 118 enable stressing the superelastic wire supports into the desired configuration while the stressed superelastic wire supports are exposed to heat ranging from 300° C. to 550° C., as previously described, and then quenched or otherwise allowed to return to ambient temperature. As previously stated, this exposure to heat and subsequent quenching of stressed materials forms the superelastic wire supports into the desired shape of the brace. The thermal forming fixture 118 may be fabricated from aluminum, stainless steel, or other alloy capable of withstanding the temperature the superelastic wire supports need to be exposed during thermal forming, and able to maintain the stressed superelastic wire supports into the desired shape.

The thermal forming fixture is fabricated to define the desired anatomic joint geometry. In the embodiment shown in FIGS. 5a to f, the thermal forming fixture is designed to fabricate knee braces. The thermal forming fixture may be cast, machined, or otherwise manufactured into the desired shape. Other thermal forming fixture configurations may be used to fabricate braces for other anatomic joints and/or with different sizes. As shown in FIG. 5g, the thermal forming fixture 118 incorporates shaping clips 120 designed to maintain the superelastic wire supports in their stressed configuration. Springs 122 secure the shaping clips 120 to the thermal forming fixture 118 such that the shaping clips 120 may be opened while positioning the superelastic wire supports into desired shape, and released to maintain the superelastic wire supports in the stressed configuration.

As shown in FIG. 5a, the superelastic wire support 60 is secured to two shaping clips 120 oriented below the knee joint to define the lower tightening link 65. As shown in FIG. 5b, the superelastic wire support 60 is curved and secured to a shaping clip 120 located adjacent the knee at the desired hinge point to define the spring link 63. As shown in FIGS. 5c and d, the superelastic wire support 60 is further curved and secured to two shaping clips 120 located above the knee. As shown in FIGS. 5e and f, the superelastic wire support 60 is further shaped to define the opposite side of the brace, which mirrors the side previously described. The superelastic wire support 60 may be shaped into the desired 3-dimensional geometry in sequential thermal forming steps or a single thermal forming step. The completed knee brace, shown in FIG. 5f, consists of two tightening links 65 and two spring links 63. The spring links 63 consist of single loops that expand in diameter as the knee joint is flexed such that the calf deflects towards the hamstring, and decrease in diameter as the knee joint is extended in the opposite direction.

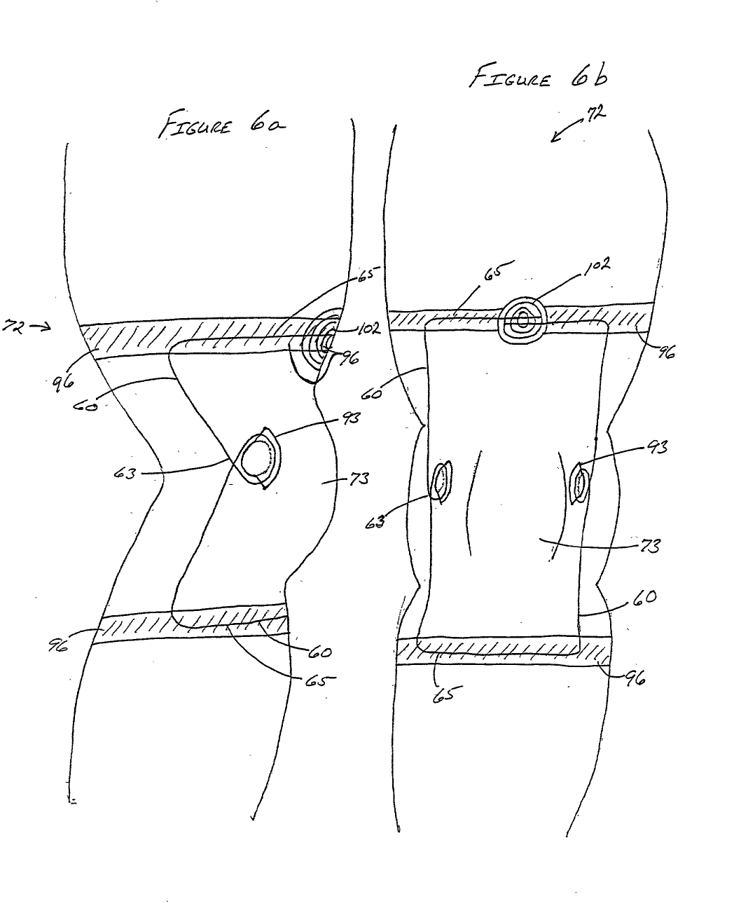

FIGS. 6a and b show a side view and a front view of the shaped superelastic wire support 60 shown in FIG. 5f incorporating 1) one interconnect locking means 102 to secure the free ends of the superelastic wire support 60 and maintain the final shape of the brace, 2) two locking mechanisms 96 to removably attach the tightening links 65 to the leg at the desired locations, and 3) two hinge stop mechanisms 93 located at the two looping spring links 63 to limit the possible reduction in diameter of the loops formed with the superelastic wire support 60 thus prevent hyperextension of the knee joint. This knee brace configuration 72 better limits abnormal rotation and translation of the knee joint while preserving natural motion of the leg.

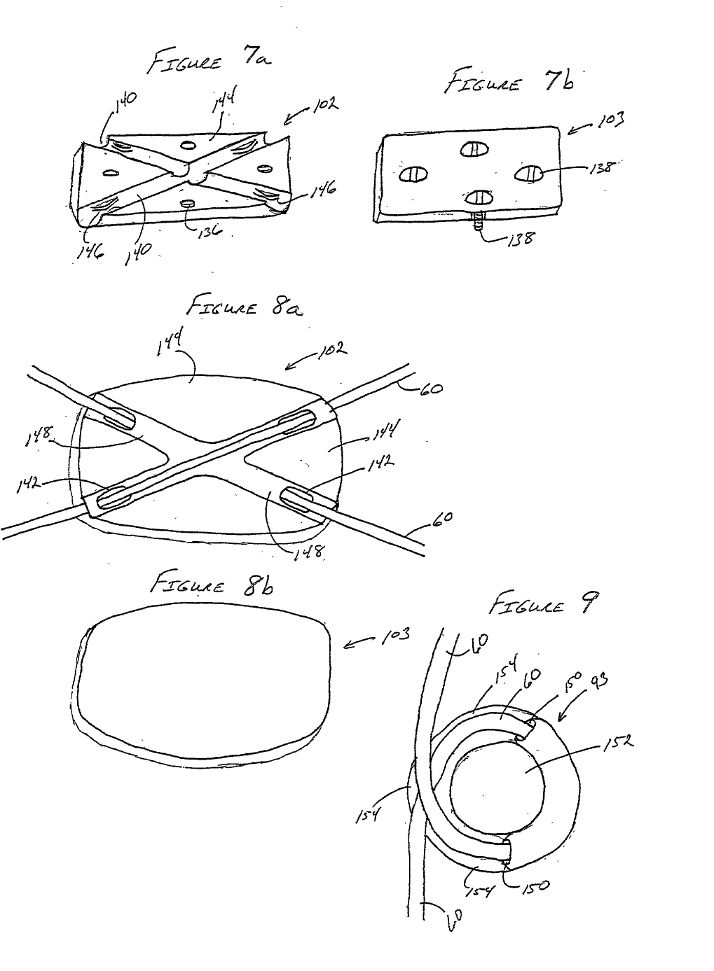

FIGS. 7a and b show the bottom piece 102 and top piece 103 of an interlocking mechanism embodiment used to secure free ends of superelastic wire support(s) 60, adjacent segments of superelastic wire support(s) 60, or intersecting segments of superelastic wire support(s) 60. The interlocking mechanism 102 incorporates a housing 144 to which all components of the interlocking mechanism are bonded, channels 140 through which segments of superelastic wire support(s) are inserted and secured, protrusions 146 to restrict sliding of the superelastic wire support(s), and threaded holes 136 to enable attaching the top piece 103 of the interlocking mechanism through the use of screws. Other bonding mechanisms to secure the top piece 103 and bottom piece 102 of the interlocking mechanism may be utilized including ultrasonic welding, thermal bonding, snap-fits, radio frequency bonding, laser welding, adhesive bonding, or other attachment mechanism. The interlocking mechanism components may be fabricated from Pebax, polyethylene, polyester, polyurethane, polycarbonate, stainless steel, silicone, nylon, a combination of materials, or other material.

The radial and axial stiffness of the superelastic wire support(s) may be modified by changing the angles that adjacent sections of the superelastic wire support(s) intersect and/or extend away from the interlocking mechanism. The positions and orientations of the channels 140 determine the angles the superelastic wire support(s) emanate from the interlocking mechanism, thus the channels 140 may be modified to change the characteristics of the superelastic wire supports. The brace characteristics may be alternatively varied by attaching additional modular interlocks to the already secured interlocking mechanisms 102. These modular interlocks fit around the interlocking mechanisms and are bonded in place to change the angles the superelastic wire support(s) extend from the interlocking mechanisms thus the radial and/or axial stiffness parameters. In addition, these modular interlocks change the lengths of the spring links and/or tightening links further changing the radial and/or axial stiffness parameters.

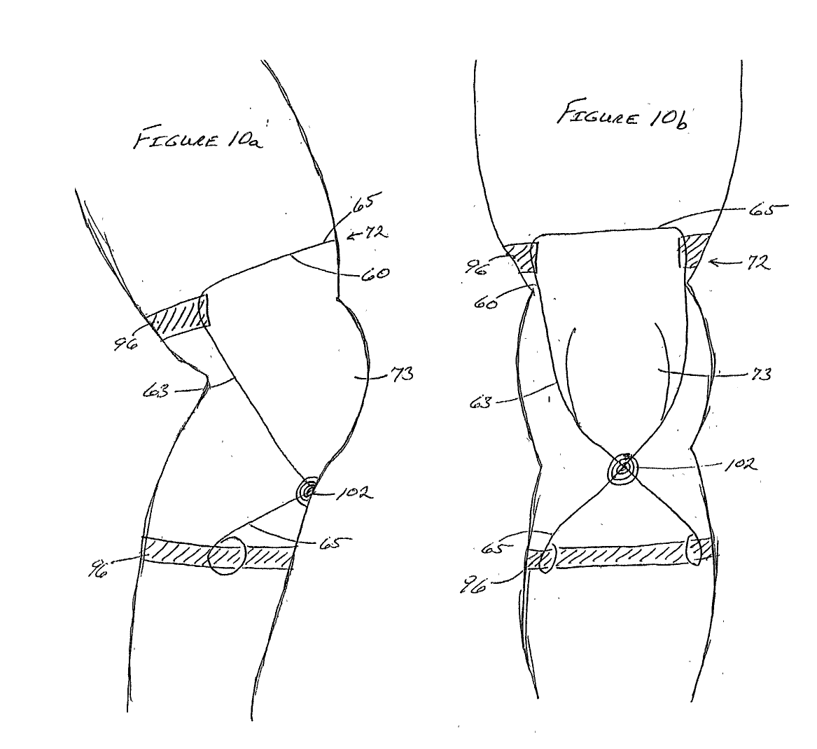

FIGS. 8a and b show an alternative interlocking mechanism 102 embodiment used to secure segments of superelastic wire support(s) 60. The interlocking mechanism 102 consists of a housing 144 onto which a feeding anchor piece 148 is bonded. The feeding anchor piece 148 contains holes 142 at predetermined locations through which superelastic wire support(s) 60 are threaded, as shown in FIG. 8a. Threading the superelastic wire support(s) 60 through holes 142 in the feeding anchor piece 148 secures the superelastic wire support(s) 60 to the interlocking mechanism 102 and prevents sliding of the superelastic wire support(s). Once the top piece 103 is bonded to the housing 144, the interconnecting mechanism 102 restricts sliding, bending, or other motion of the segments of superelastic wire support(s) enclosed in the interconnecting mechanism 102.

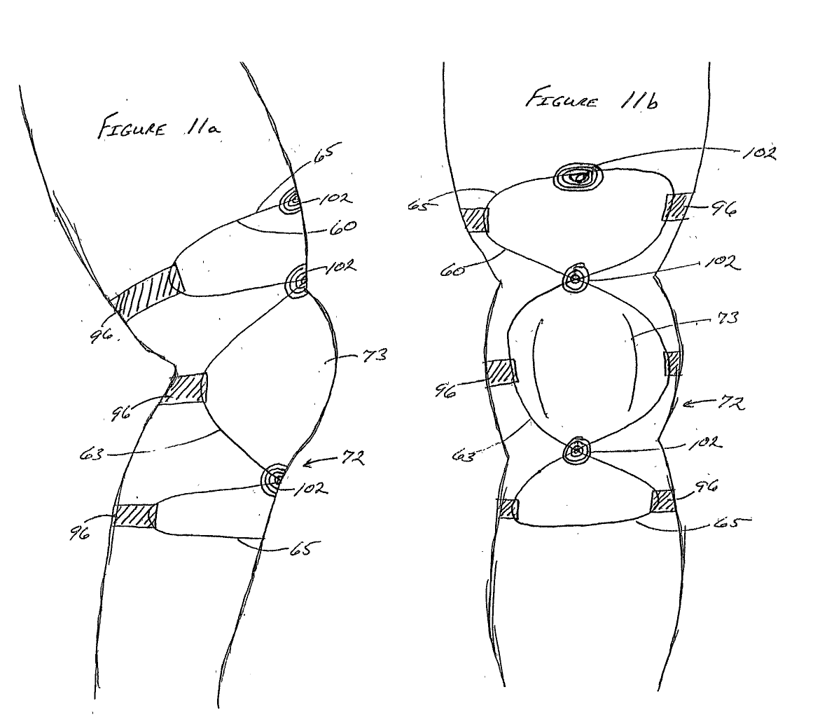

FIG. 9 shows a hinge stop mechanism 93 designed to prevent hyperextension of the knee by restricting movement of each looping spring link 63 in the knee brace embodiment shown in FIGS. 6a and b. The hinge stop mechanism 93 incorporates a housing 154 through which a channel 150 extends partially around the looping spring link 63. A looping section of the superelastic wire support resides through the channel 150. A protrusion 152 extending from the housing 154 and having a desired diameter determines the minimum radius of curvature the looping spring link 63 is allowed to deflect. As previously discussed, the looping spring link 63 is fabricated by winding the superelastic wire support 60 into at least one loop. The hinge stop mechanism 93 allows expansion of the loop diameter and prevents reduction in the diameter of the superelastic wire support(s) 60 beyond the diameter of the protrusion 152 thereby introducing directionality in the deflection of the superelastic wire support(s) 60. The hinge stop mechanism components may be manufactured from Pebax, polyethylene, polyester, polyurethane, polycarbonate, nylon, stainless steel, or other material.

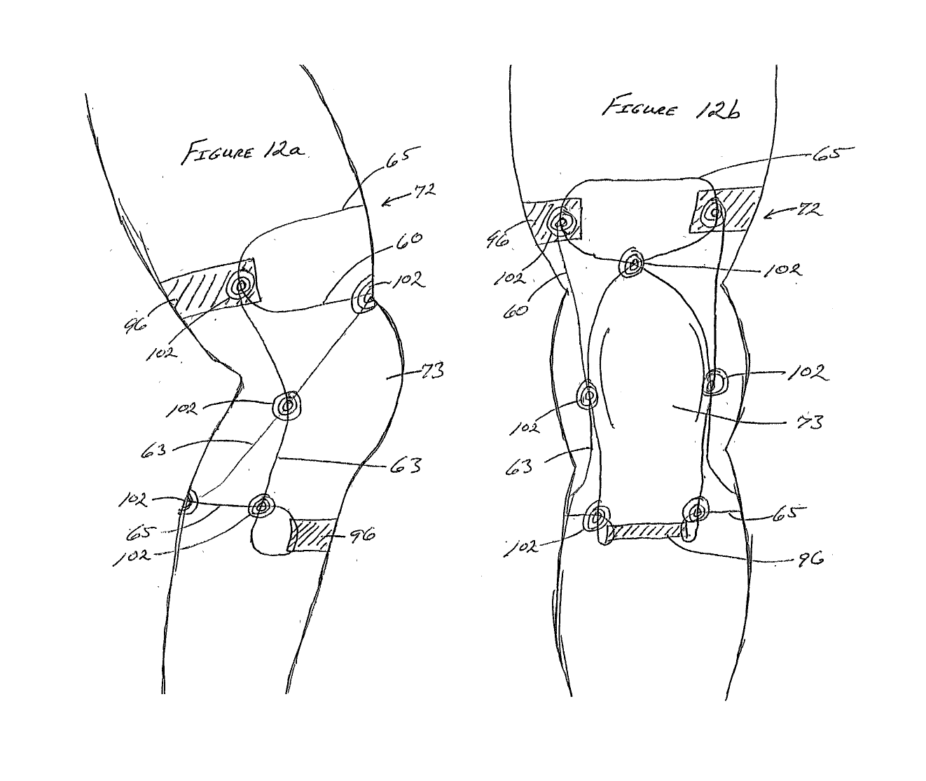

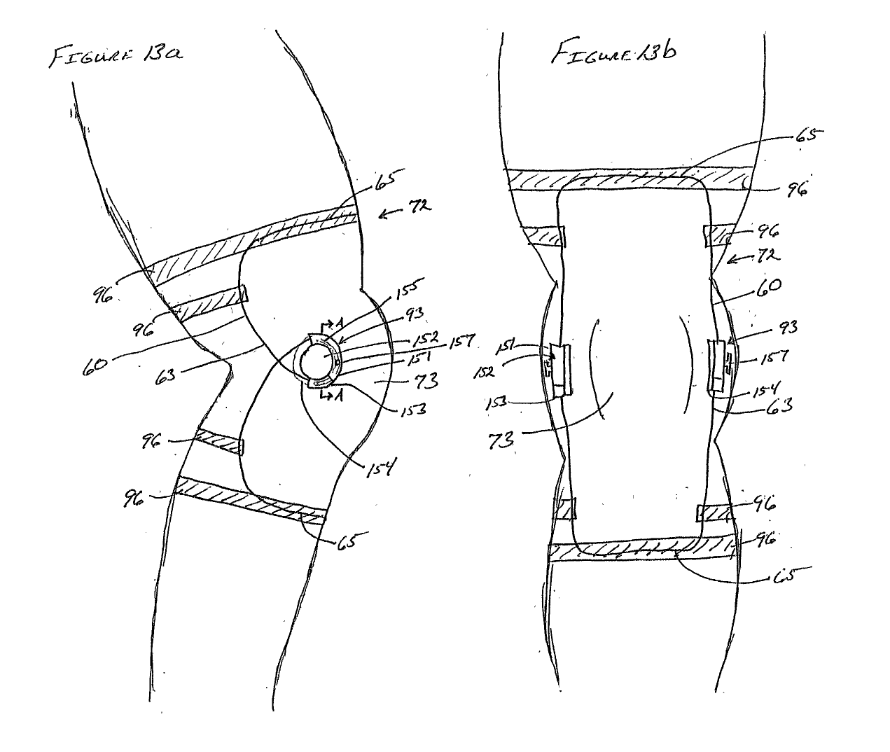

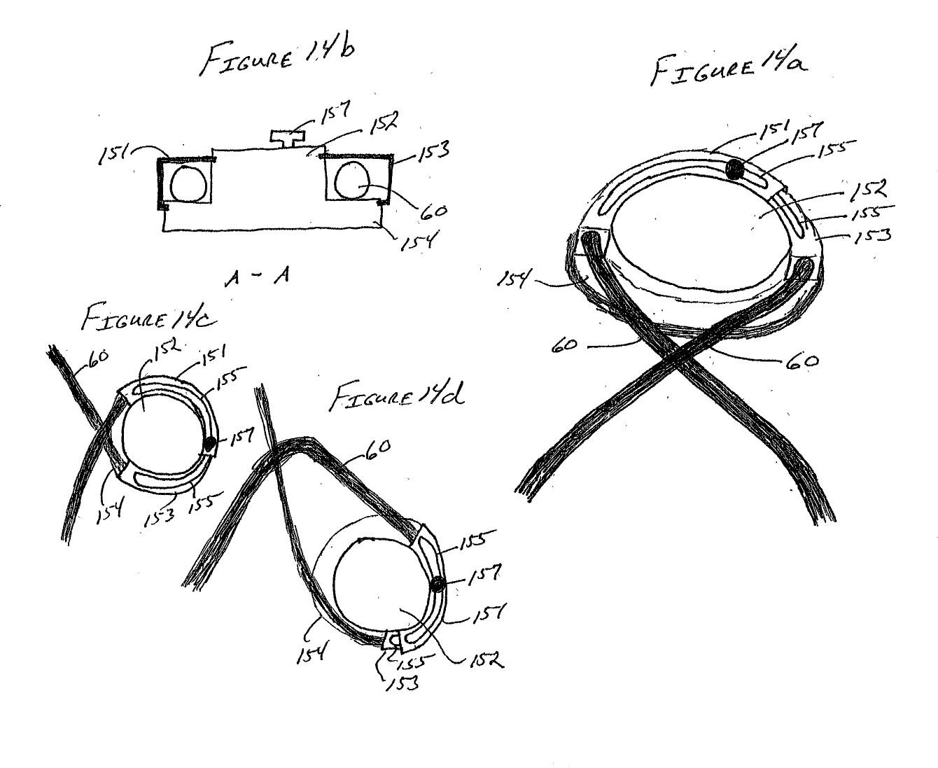

FIGS. 10a and b, 11a and b, and 12a and b show side views and front views of alternative knee brace embodiments 72 that incorporate attachment mechanism 96 and interlocking mechanisms 102. The knee brace embodiments may be fabricated for specific applications by defining the axial flexibility and radial stiffness to the desired motion of the leg about the knee joint.

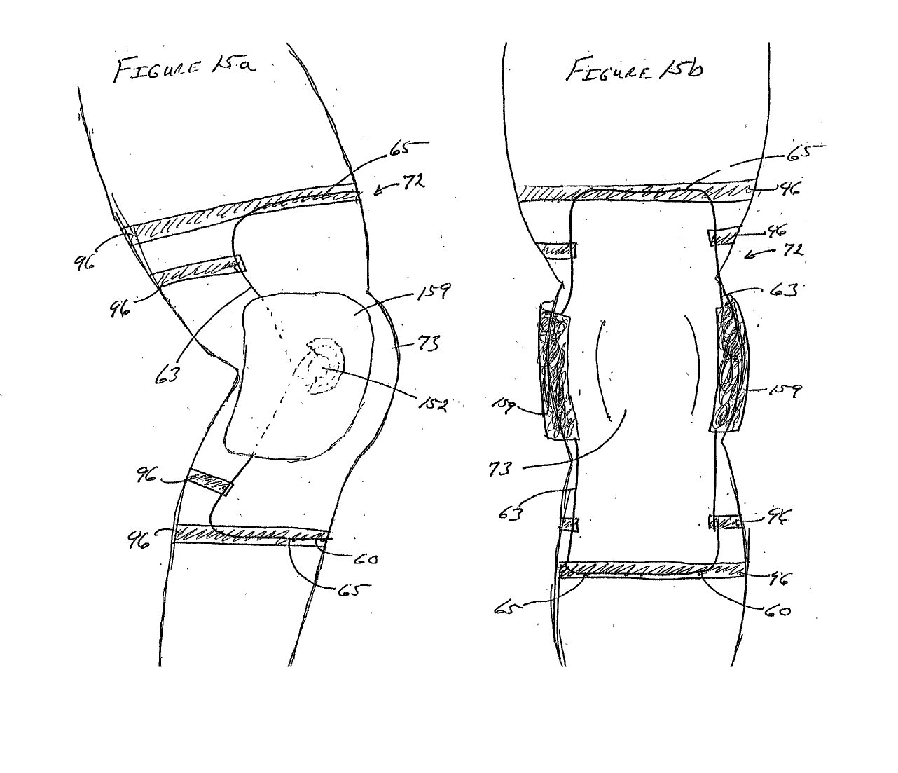

FIGS. 13a and b show a side view and a front view of a knee reinforced by a knee brace 72 fabricated from at least one superelastic support (preferably, but not limited to, a wire or rod) 60. This knee brace 72 embodiment incorporates at least one superelastic wire or rod support (in this case a single wire or rod having a diameter between 0.040″ and 0.300″ is used) thermally formed into the desired 3-dimensional geometry designed to support the knee and produce an integrated hinge restricting the motion of the joint to a desired tract. The distribution and the characteristics of the superelastic wire support(s) determine the purpose for the brace and the amount of force the brace is capable of exerting in response to an external force such as a deflection. The superelastic wire supports have at least one diameter, D, for circular wires (a major axis and a minor axis dimension for elliptical, oval, rectangular, trapezoidal, or other geometry wires), and at least one length configured to produce the desired stiffness and force profile. The diameter and/or length may vary throughout the superelastic wire supports to vary the stiffness profile and resulting response to movement.

The knee braces in FIGS. 13a and b incorporate two tightening links 65 connected with two opposing spring links 63. The spring link 63 defines the integrated hinge, which permits flexion of the knee and determines the response of the brace to such flexion. During manufacture the cross-section of each superelastic support link may be a circular rod, a rectangular band, a circular or elliptical wire, a square ribbon, a donut shaped tube, or other geometry that provides the desired stiffness to impart the reinforcing and spring forces; in this embodiment, the superelastic support links are a circular or elliptical wire wrapped into the desired pattern. It should be noted that the orientation of the superelastic support links relative to the leg depends on the purpose for the brace and helps dictate the restriction of abnormal motion and the spring characteristic of the brace. The embodiment in FIG. 13a has two spring links 63, one on each side of the leg; each spring link 63 extends from a tightening link 65 located above the knee 73 to a tightening link 65 below the knee. The spring links 63 permit the desired amount of movement about the joint and exert a desired amount of elastic recoil in response to an induced deflection. It should be noted that any number of spring links might be chosen depending on the manufacturing process, the desired spring constant, and the desired stiffness profile.

FIGS. 14a to d show a hinge stop mechanism 93 designed to prevent hyperextension of the knee by restricting movement of each looping spring link 63 in the knee brace embodiment shown in FIGS. 13a and b. The hinge stop mechanism 93 incorporates a housing 154 through which two independently movable channels 151 and 153 extend partially around the looping spring link 63. A looping section of the superelastic wire support resides through the channels 151 and 153. The relative positions of the channels 151 and 153 to each other determine the angle at which the looping section of the superelastic wire support extend from the hinge stop mechanism 93, which determines the degree of extension the knee is able to be exposed. A screw 157 is used to affix the relative positions of the channels 151 and 153. A protrusion 152 extending from the housing 154 and having a desired diameter determines the minimum radius of curvature the looping spring link 63 is allowed to deflect. As previously discussed, the looping spring link 63 is fabricated by thermally forming the superelastic wire support 60 into at least one loop. The hinge stop mechanism 93 allows expansion of the loop diameter and prevents reduction in the diameter of the superelastic wire support(s) 60 beyond the diameter of the protrusion 152 thereby introducing directionality in the deflection of the superelastic wire support(s) 60. The hinge stop mechanism components may be manufactured from Pebax, polyethylene, polyester, polyurethane, polycarbonate, nylon, stainless steel, or other material.

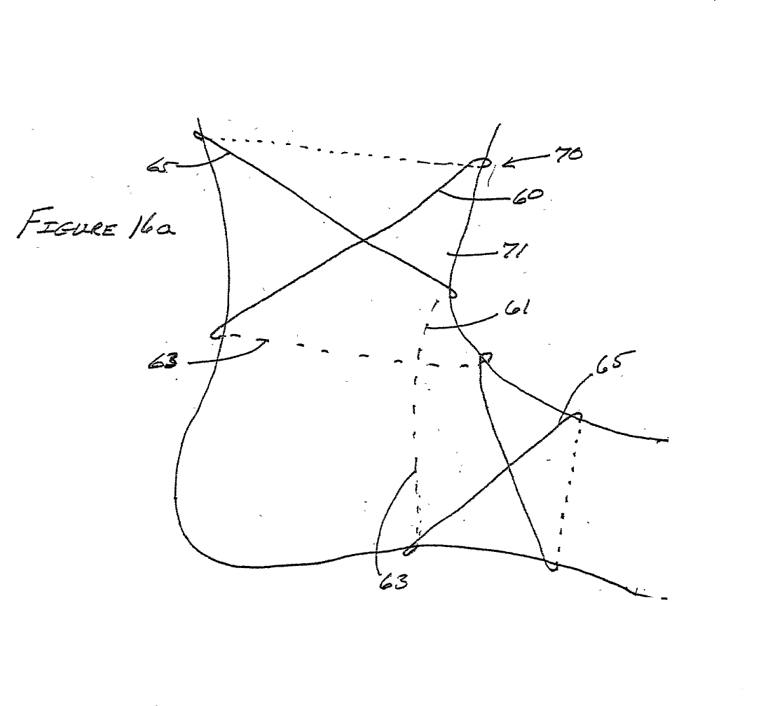

FIGS. 15a and b show a side view and a front view of an alternative knee brace embodiment 72 that further incorporates padding 159 along the sides of the brace. The padding covers the spring link and hinge stop mechanism, if present, and protects the sides of the knee from direct impact. The padding may alternatively be fabricated to also cover the patella providing additional reinforcement of the knee structures.

The superelastic wire support structures previously described for the knee braces above may additionally be modified accordingly for back traction devices, ankle braces, elbow braces, wrist braces, shoulder braces, hip braces, neck braces, other joint braces, or other anatomic scaffolds. Similarly, the superelastic wire support structures described below for knee braces, and traction devices for the back may be modified for other anatomic joints or structures.

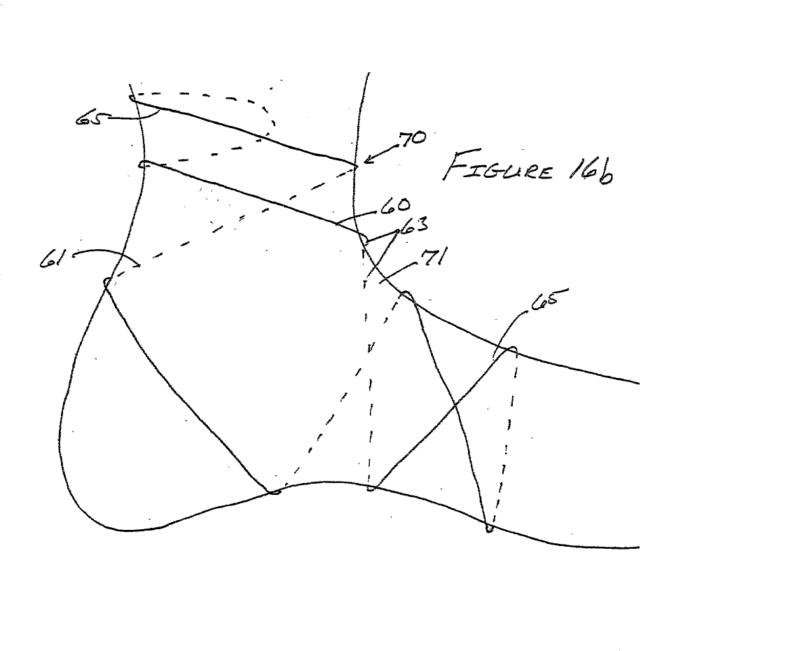

FIGS. 16a to c show ankle braces that contain superelastic supports. As previously described, the ankle brace may incorporate at least one superelastic support 60 embedded in at least one covering (not shown). As previously described, the covering(s) may embed individual links of the superelastic supports, a subset of the superelastic supports, or the entire superelastic support structure. The superelastic support structures 60 in FIGS. 16a to c contain two tightening links 65, one located somewhere along the shin (or other location proximal to the ankle), and one located at the arch of the foot (or other location distal to the ankle). The tightening links 65 are connected with at least one spring link 63. The embodiment shown in FIG. 16a consists of a first tightening link 65 located at the shin and comprising a wire of superelastic material formed in a crisscross on one side of the leg and in horizontal, relatively parallel lines on the opposite side of the leg; a second tightening link 65 located at the arch of the foot and comprising a wire of superelastic material formed in a crisscross on one side of the foot and in vertical, relatively parallel lines on the opposite side of the foot; a first spring link 63 connecting the criss-cross for the proximal tightening link 65 to the vertical extension of the distal tightening link; and a second spring link 63 connecting the criss-cross for the distal tightening link 65 to the horizontal extension of the proximal tightening link. The spring links 63 for this embodiment may be located on either the outside surface or the inside surface of the ankle. The ankle brace 70 in this embodiment and the alternative embodiments described below prevent rolling (twisting) of the ankle, especially when the inside surface of the foot lands on a raised object, which would cause a non-reinforced ankle to twist outward.

The spring links 63 of this ankle brace 70 embodiment and the alternative embodiments described below, may be thermally formed so that the resting position of the superelastic support is such that the balls of the foot are bent towards the front of the leg, the balls of the foot are bent away from the front of the leg, or at some position in between. The preshaped orientation of the ankle brace depends on the activity. For jumping, walking, and running, the superelastic supports are thermally formed such that the balls of the foot are bent away from the front of the leg so the spring links 63 tend to urge the foot in a downward position. The weight of the athlete wearing the ankle brace causes the spring links 63 to deflect as the balls of the foot are deflected upward toward the front of the leg, producing a spring force. Once the wearer of the ankle brace begins to remove or reduce the deflection force on the spring links 63 by pushing against a surface with the balls of the foot, the spring links 63 induce an additional downward force enhancing the jumping, walking, or running motion. For training purposes, the superelastic supports may be thermally formed such that the spring links 63 urge the balls of the foot midway between the upward and downward position or upward towards the front of the leg to provide a resistance against the desired motion of the foot. As a result, the superelastic supports oppose downward deflection of the foot making the wearer expel additional energy and force to jump, walk, and/or run thereby aiding the development of the muscles relied upon to jump, walk, and/or run.

FIG. 16b shows an alternative ankle brace embodiment. This ankle brace 70 consists of a first tightening link 65 located at the shin and consisting of wires or other superelastic material configuration coiled around the leg in relatively parallel lines that are interconnected with a loop; a second tightening link 65 located at the arch of the foot and consisting of a criss-cross on one side of the foot and vertical, relatively parallel extensions on the opposite side of the foot; a first spring link 63 that connects one of the vertical extensions of the second tightening link to one of the relatively parallel extensions of the first tightening link; and a second spring link 63 that extends from the criss-cross of the second tightening link, around the heel of the foot and to the other relatively parallel extension of the first tightening link 65.

The ankle brace embodiment in FIG. 16c comprises a proximal tightening link 65 connected to a distal tightening link 65 by two spring links 63 located on one side of the ankle. The ankle braces described above illustrate potential embodiments for the orientation of tightening links 65 and spring links 63; any number and/or combination of tightening links and spring links that prevent twisting or over-bending the ankle joint, and induce an opposing force in response to a deflection may be utilized.

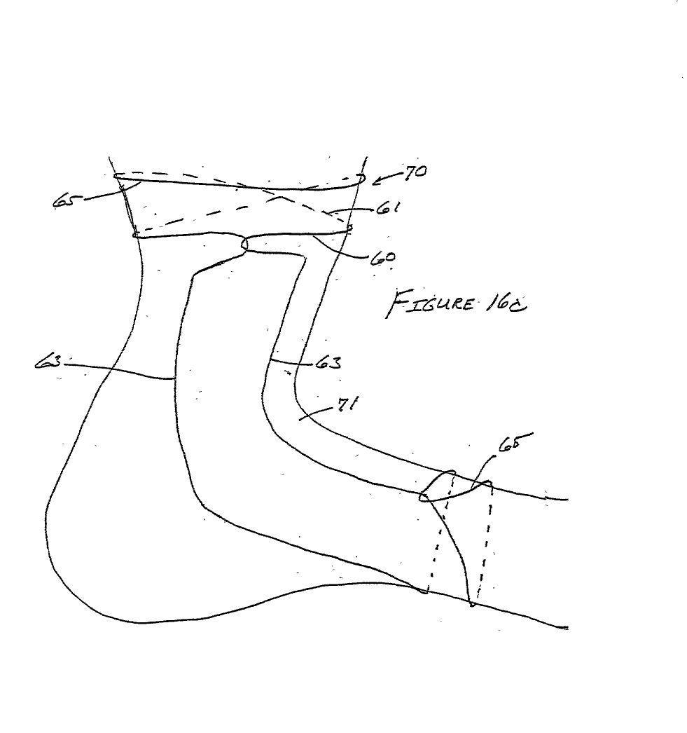

The support member structures previously described for the knee and ankle braces may additionally be modified accordingly for elbow, wrist, shoulder, back, hip, other joint braces, or other anatomic scaffolds. FIG. 17 shows an elbow brace 74 that incorporates a superelastic support structure 60 that reinforces the elbow joint. This superelastic support structure 60 embodiment prevents over-rotating, excess twisting, unwanted bending or separation of the tissues at the elbow joint 75. The superelastic support structures 60 of the elbow brace 74 also direct the motion of the forearm depending on the activity. For throwing motions, the superelastic support structures 60 of the elbow brace 74 enhance bending the forearm at the elbow joint 75 and prevent excess rotation of the forearm. As such, the superelastic support structures of the elbow braces enable directing motion of the forearm in the correct or optimal form (depending on the activity), prevent injury of the muscles and tendons at the joint due to dramatic over-stressing or chronic fatiguing, and increase the applied force of the motion by providing an elastic recoil of the superelastic support structure due to a deflection force. The elbow brace may alternatively be fabricated extremely stiff for activities where bending at the elbow is minimal but supporting a heavy object with the arms is required. This applies to shooting activities, archery, and other endeavors where the optimal positioning of the forearm at the elbow joint may be enhanced by a stabilizing support structure that prevents or limits motion.

The elbow brace 74 in FIG. 17 incorporates a first tightening link 65 located at the biceps, a second tightening link 65 located along the forearm, two spring links 63 located on one side of the arm and interconnecting the first and second tightening links, and three spring links 63 located on the other side of the arm and interconnecting the tightening links, and the other spring links. It should be noted that the two sets of spring links 63 for this embodiment permits a specific degree of rotation about the elbow, depending on the activity, and provides additional spring force to aid the rotation in response to a deflection force. This is essential to optimize the motion of the arm structures at the elbow during activities such as throwing, hitting balls with a tennis racket, or other endeavors associated with a degree of rotation.

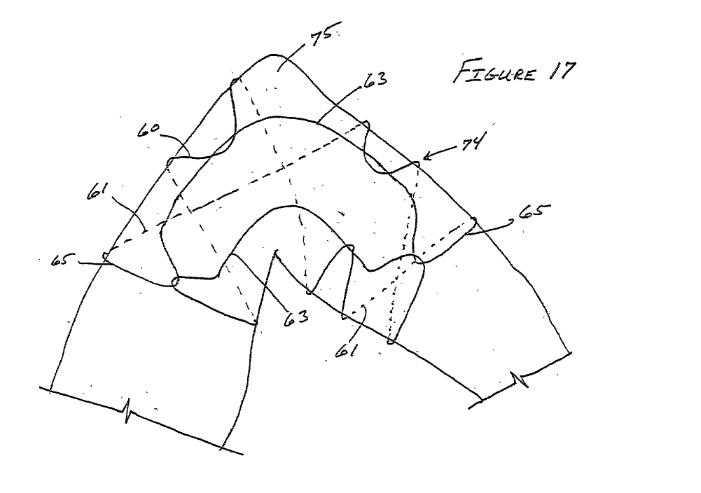

FIGS. 18a to c show wrist braces 76 that direct the motion of the hand relative to the forearm to optimize the movement of the wrist during specific activities, increase the force exerted at the wrist during activities, prevent unwanted twisting or bending at the wrist, prevent repetitive stressing of the anatomic structures, or limit motion of the hand to prevent or treat Carpal Tunnel Syndrome. The wrist brace 76 embodiment shown in FIG. 18a consists of a first tightening link 65 located proximal to the wrist 77, at the forearm; a second tightening link distal to the wrist 77 and containing a superelastic material in a criss-cross pattern with a horizontal link that extends around the palm of the hand distal; and two spring links 63 connecting the first and second tightening links.

This wrist brace may be configured by specifying the stiffness of the spring links to prevent or restrict motion of the hand towards the front or back of the forearm and direct the motion of the hand towards the sides of the forearm. As such, this motion may be configured to optimize the movement of the wrist during activities such as golf where the hand is moved to a 90 degree angle relative the side of the forearm during the back swing and is extended to the straightened, ready position at contact between the golf club and the ball. The spring link 63 enables deflection of the hand at the wrist during the backswing and induces a spring force to urge the hand towards the ready position during the downswing to cause the wrist to straighten and improve the golf swing. This wrist brace also prevents collapsing of the wrist toward the front or back of the forearm during the swing, which is commonly associated with slicing or hooking the ball.

This wrist brace may alternatively be configured to enable the hand to deflect towards the back or front surface of the forearm and prevent or restrict movement of the hand to the sides of the forearm. This may be accomplished by making the spring links 63 more rectangular or elliptical or altering the stiffness of the spring links to direct the available motion of the superelastic supports. As such this wrist brace may be suited to the shooting motion during basketball, the throwing motion during baseball or softball, or other activity mainly involving motion of the hand toward the front and/or back of the forearm. As previously stated, the spring links 63 are able to enhance the motion of the hand by increasing the applied force exerted in response to a deflection at the wrist during shooting, throwing or other activity.

FIG. 18b shows an alternative wrist brace 76 embodiment. This embodiment consists of a first tightening link 65 located on the forearm proximal to the wrist; a second tightening link located at the thumb; a third tightening link located at the middle finger; a first spring link 63 connecting the first tightening link to the second tightening link; a second spring link 63 connecting the first tightening link to the third tightening link, and a third spring link connecting the second tightening link to the third tightening link.

FIG. 18c shows another wrist brace 76 embodiment. This embodiment consists of a first tightening link 65 located on the forearm proximal to the wrist; a second tightening link located along the palm of the hand; two spring links 63 connecting the first and second tightening links 65. This configuration is more amenable to motion of the hand towards the back and front of the forearm since the spring links are located near the lateral sides of the hand.

The stiffness of the spring links 63 in the wrist brace embodiments described above may be tailored to enable a specific degree of rotation at the wrist activities involving rotation of the hand relative to the forearm. Such motion may benefit activities such as bowling where a specific degree of rotation of the hand relative to the forearm is essential to spin the bowling ball. However, the wrist brace embodiments described above are configured to prevent over-rotating and over-bending at the wrist.

The wrist braces described above may be used to treat or prevent Carpal Tunnel Syndrome associated with chronic stressing of the wrist during activities such as typing, etc. The wrist brace 76 may be configured to prevent or restrict motion of the hand relative to any surface of the forearm, reinforce the wrist to hold the position of the hand relative to the forearm without having to stress the muscles and tendons at the wrist, and maintain freedom to move the fingers.

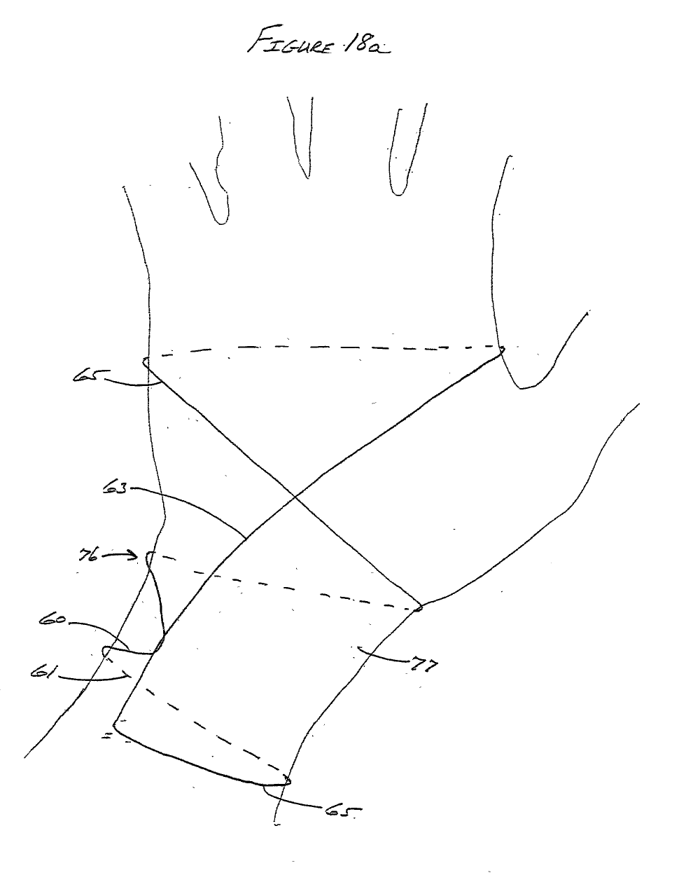

FIGS. 19a and b show shoulder brace embodiments that direct motion at the rotator cuff 79 and enhance activities by inducing a force in response to an opposing deflection. The shoulder brace embodiments 78 in FIGS. 19a and b consist of first tightening links 65 located along the shoulder proximal to the rotator cuff 79; second tightening links 65 located along the arm distal to the rotator cuff 79; first spring links 63 on one side of the shoulder connecting the first tightening links to the second tightening links; and second spring links located on the opposite side of the shoulder connecting the first tightening links to the second tightening links. These shoulder braces 78 direct rotation of the arm at the rotator cuff, prevent over-rotating or bending of the arm relative to the shoulder, and impart an induced force during an activity in response to a deflection of the arm about the rotator cuff. Alternatively, the shoulder brace 78 may restrict or prevent motion of the arm about the rotator cuff to enable healing of injured muscles, tendons, or other tissues. In addition, the spring links 63 of the shoulder braces 78 may be configured to provide a resistance during training or rehabilitation to strengthen the muscles required during an activity.

Superelastic wire (or other geometry) supports are also applicable to back braces. The superelastic wire supports may be shaped and secured at discrete locations to vary and tailor the stiffness profile of the back brace to the desired application. The benefits of superelastic wire supports in back braces over rigid devices is that the superelastic wire supports enabled controlled motion of the spine yet reinforces the back during movement. The spring force induced by deflection of the superelastic wire supports facilitates correct posture by straightening the back after the brace wearer bends down to pick up an object, bends at the waist to sit, or otherwise moves in a direction the wearer eventually and controllably desires to reverse. Other back brace designs are unable to apply a spring force in response to a deflection and are extremely expensive since they require several inflatable bladders that are filled with air, fluid or pads that are tightened with elastic bands. Superelastic wire supports are inexpensive and accommodate the variability in stiffness required for back braces.

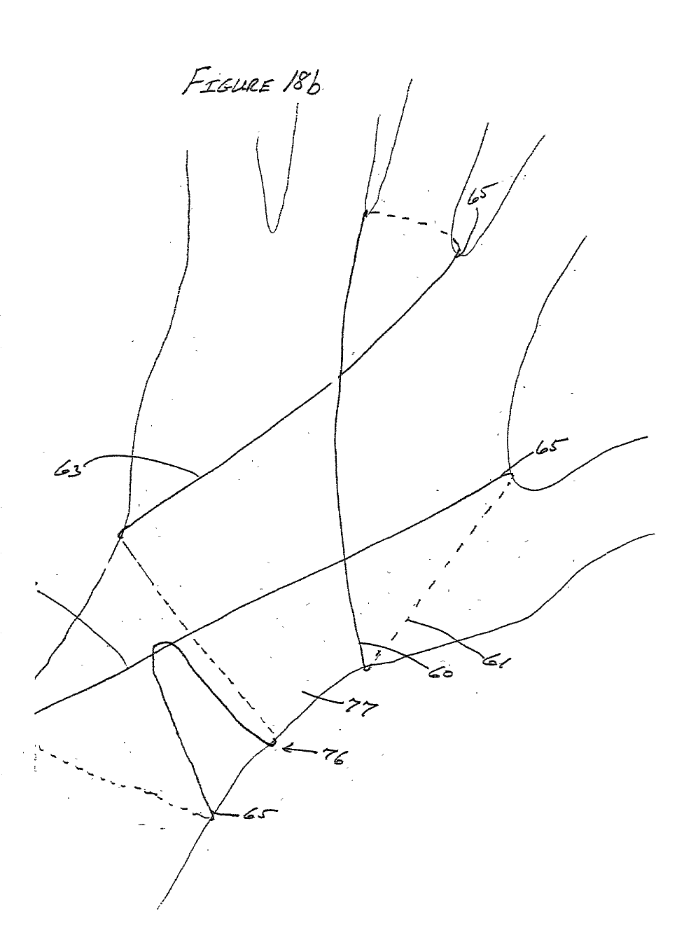

FIGS. 20a to c show back braces 80 that consist of combinations of tightening links 65 and spring links 63 designed to direct rotation and bending of the back relative to the hips. These back braces reinforce the back muscles to prevent spasms or injuries to the back, and enhance the movement of the back to induce an additional force during an activity.

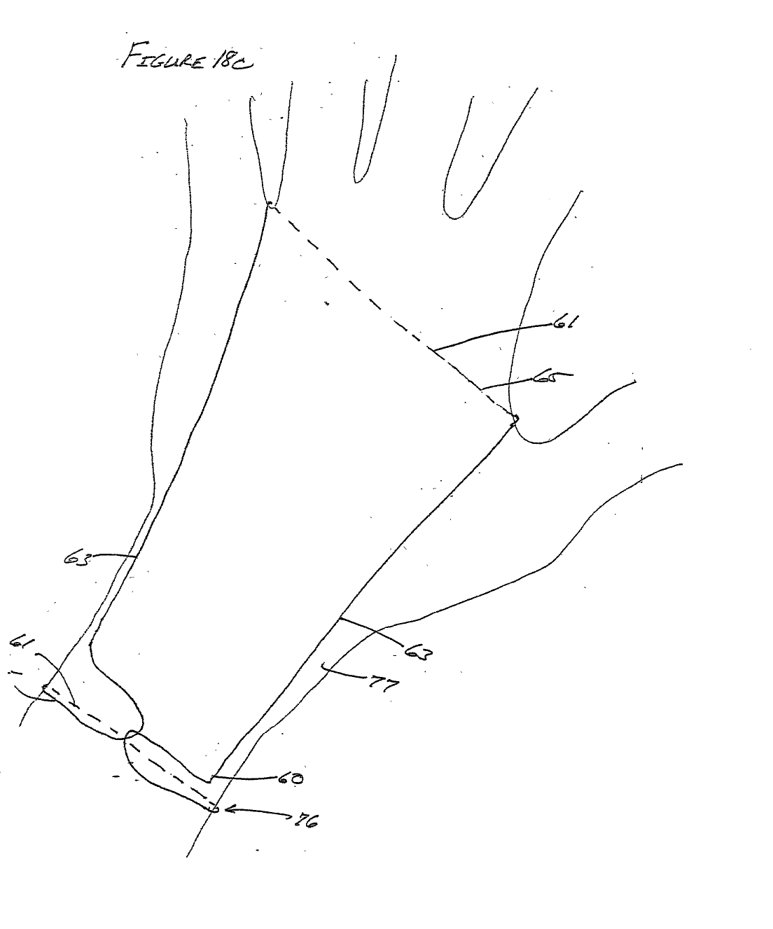

FIGS. 21a and b show two back brace embodiments 80 that consist of combinations of tightening links 65 and spring links 63 designed to direct rotation and bending of the back relative to the hips. These back braces reinforce the back muscles and spine to prevent spasms or injuries to the back, and enhance the movement of the back to induce an additional force during an activity. The spring links 63 of these back brace embodiments induce a spring force designed to counteract gravity and decrease the stress on the back while straightening the back from a bent orientation. The back brace embodiments in FIGS. 21a and b consist of single superelastic wire supports 60 wound into the pattern of tightening links and spring links shown. Of course, multiple superelastic wire supports 60 may alternatively be used to form the brace structure. Interlocking mechanisms 102 attach the free ends of the superelastic wire support 60 to the spring links 63. An attachment mechanism 96 is bonded to opposite sides of the brace and incorporates a removable anchoring means that enables tightening, securing, and removing the back brace. The anchoring means of the attachment mechanism may consist of Velcro, a ratcheting means, a buckle, a screw-type mechanism, or other temporary and adjustable securing device.

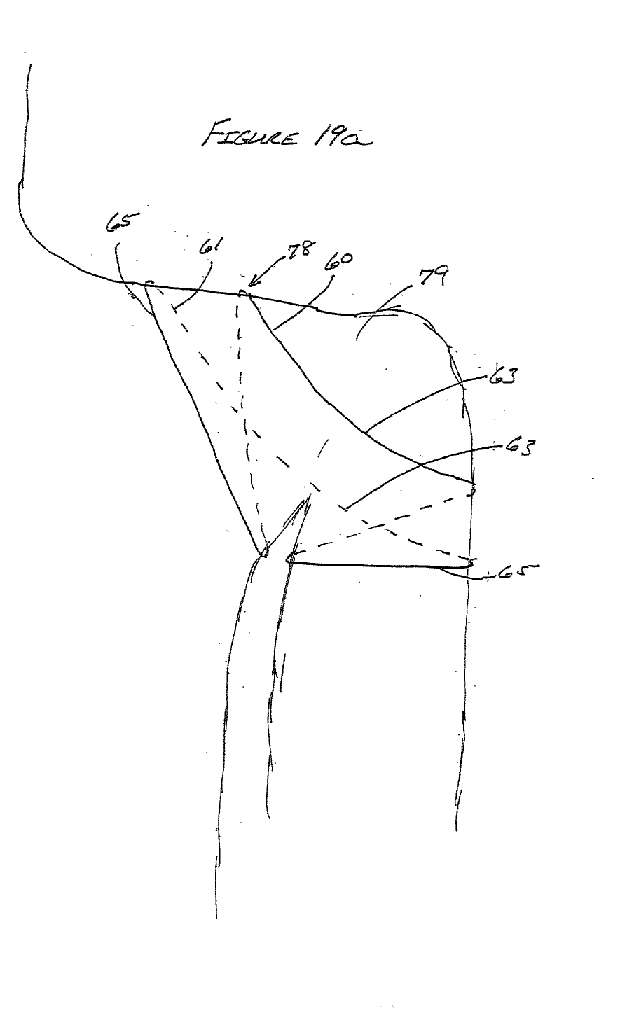

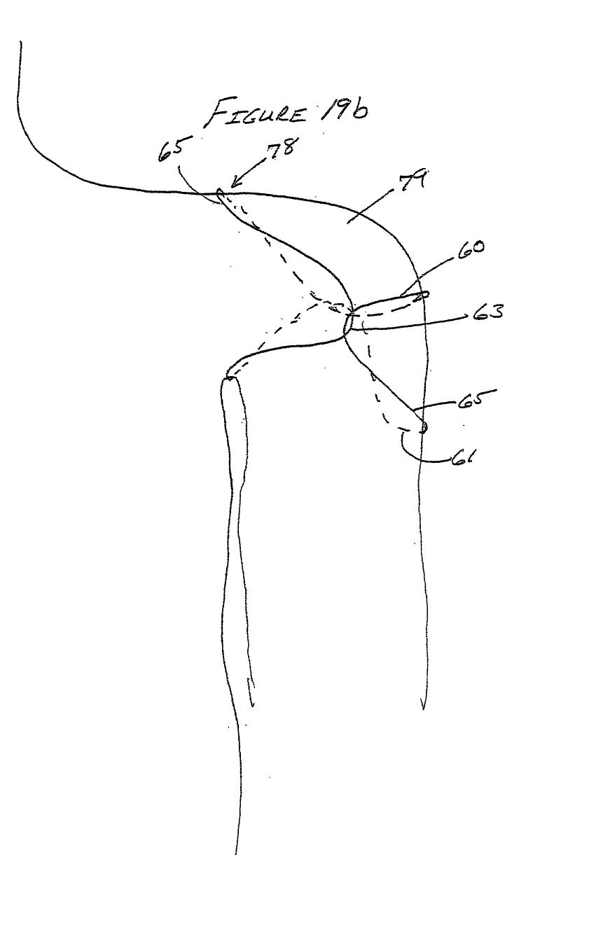

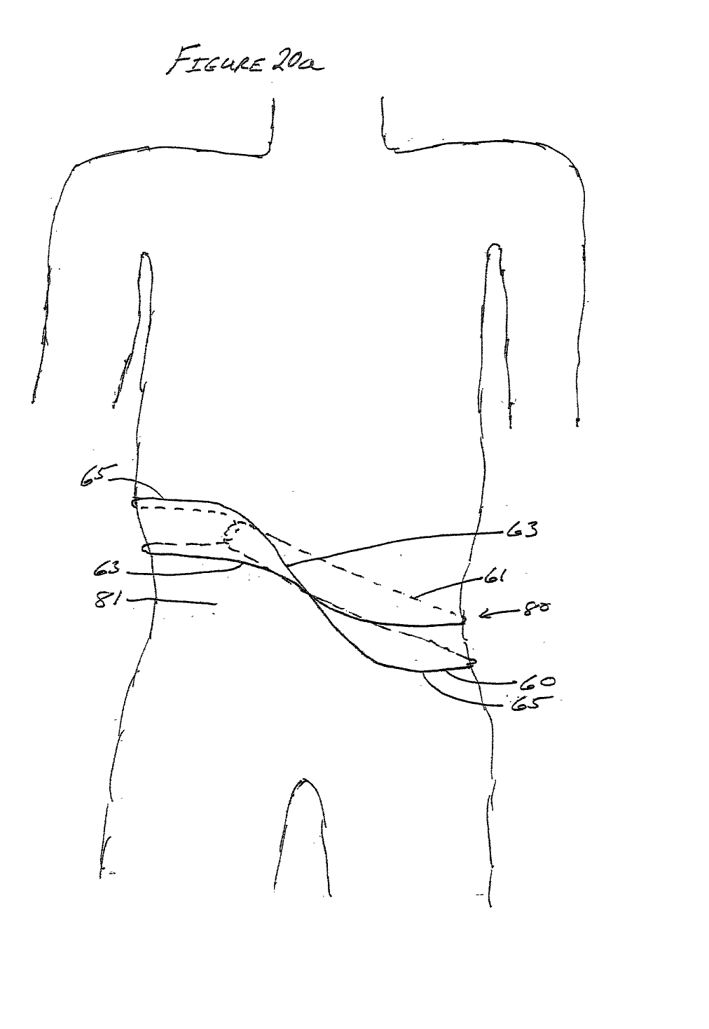

FIGS. 22a and b show an alternative back brace embodiment fabricated as a belt containing pockets at desired locations. The belt is fabricated from polyester, cotton, Kevlar, or other fabric or material woven and sewn into the desired shape and pattern of pockets and anchoring mechanisms. The belt may or may not be elastic. Sections of superelastic wire supports 60 may be inserted into the pockets 128 to reinforce sections of the back brace 80 and provide the desired stiffness and spring profile throughout regions of the brace 80. The use of pockets enables varying the properties of the back brace by inserting individual superelastic wire supports 60 having various stiffness characteristics, shapes, or other features.

FIG. 22c shows another back brace embodiment fabricated as a belt. This back brace 80 incorporates at least one superelastic wire support 60 formed in a sinusoidal pattern having various heights (h1 and h2 shown in FIG. 22c). The change in heights accommodates changes in anatomy (such as shorter heights to fit below the ribcage). Changing heights also accommodates differences in desired surface area reinforcement, spring characteristic, or stiffness. This back brace 80 incorporates at least one covering layer 127 encompassing the at least one superelastic wire support 60. The back brace 80 incorporates a first set of locking mechanisms 134 to attach the brace at the front and a second set of locking mechanisms 134 to tighten the elastic bands used to change the properties of the superelastic wire supports 60. Shoulder straps 124 stabilize the back brace to the shoulders and shoulder strap locks 126 enable altering the shoulder straps 124 to accommodate various patient anatomical shapes.

FIGS. 22d to i show various cross-sections of the back brace embodiments shown in FIGS. 22a to c taken along line A-A. The back braces incorporate at least one superelastic wire support 60 formed into the desired cross-sectional shape and encapsulated, sewn, adhesively bonded, or otherwise secured to a covering 127, if needed or wanted. The configuration in FIG. 22d accommodates indentations throughout the back such as the space between the buttocks and the middle region of the spine. This configuration may be reversed to accommodate protrusions such as the region between the lower back and the upper back. FIGS. 22e shows a straight cross-section for back brace regions that accommodate flat regions of the back, sides, or front of the body. The cross-section in FIG. 22f characterizes back brace regions that accommodate protrusions such as the ribcage. FIGS. 22g and h show cross-sections of back brace regions that provide cushioning, or apply continuous pressure by exerting a spring force against the back regions. FIGS. 22i show the cross-section of a back brace region that accommodates an indentation such as the region between the buttocks and the middle region of the spine. This configuration may be reversed to accommodate protrusions such as the region between the lower back and the upper back. These cross-sections may be incorporated in any back brace embodiment by preshaping the superelastic wire support to provide cushioning or reinforce anatomy at anatomical protrusions, indents, or other nonlinearities. Multiple cross-sectional profiles may be utilized in a single back brace embodiment to accommodate the anatomy of the body and the differing stiffness and spring requirements throughout the body.

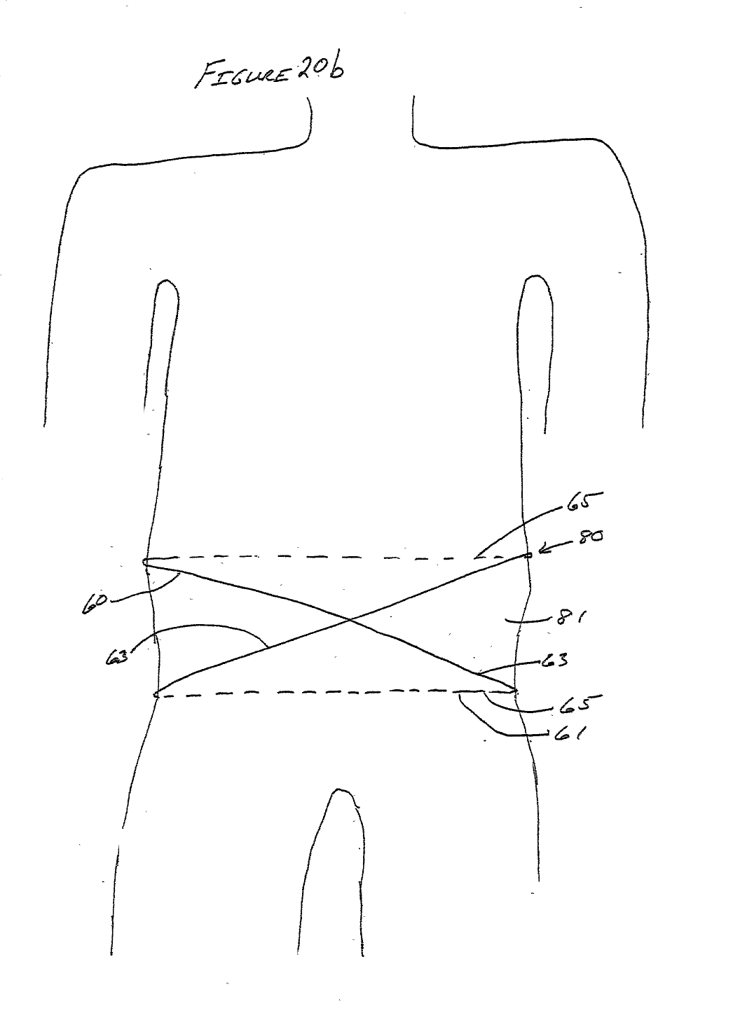

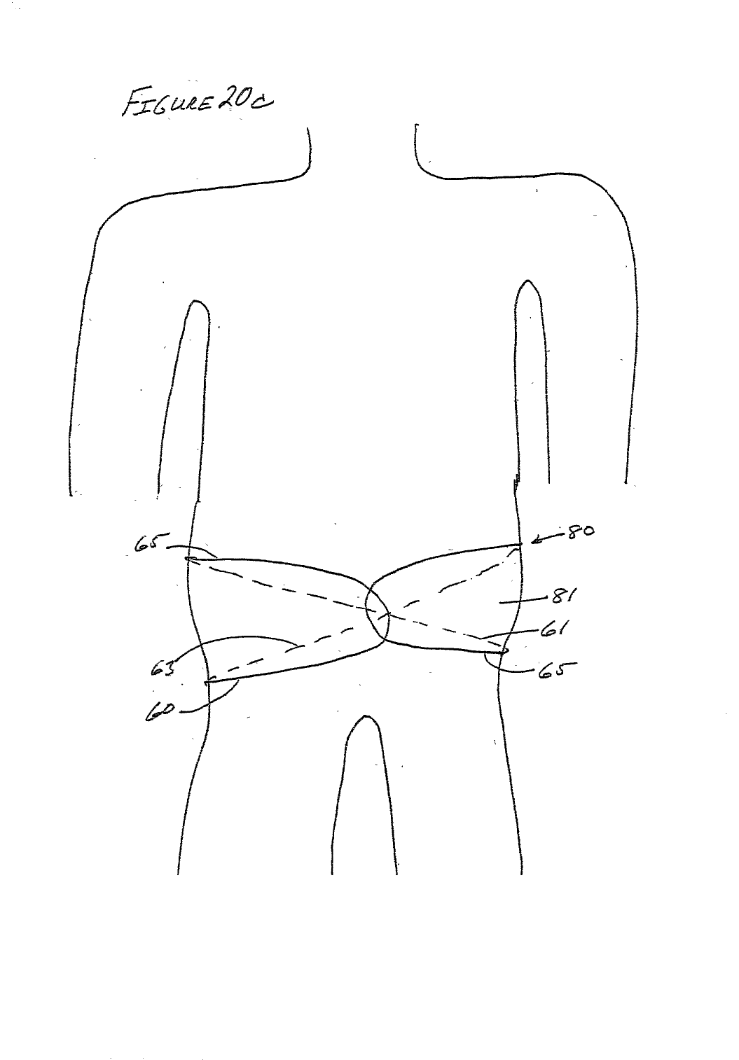

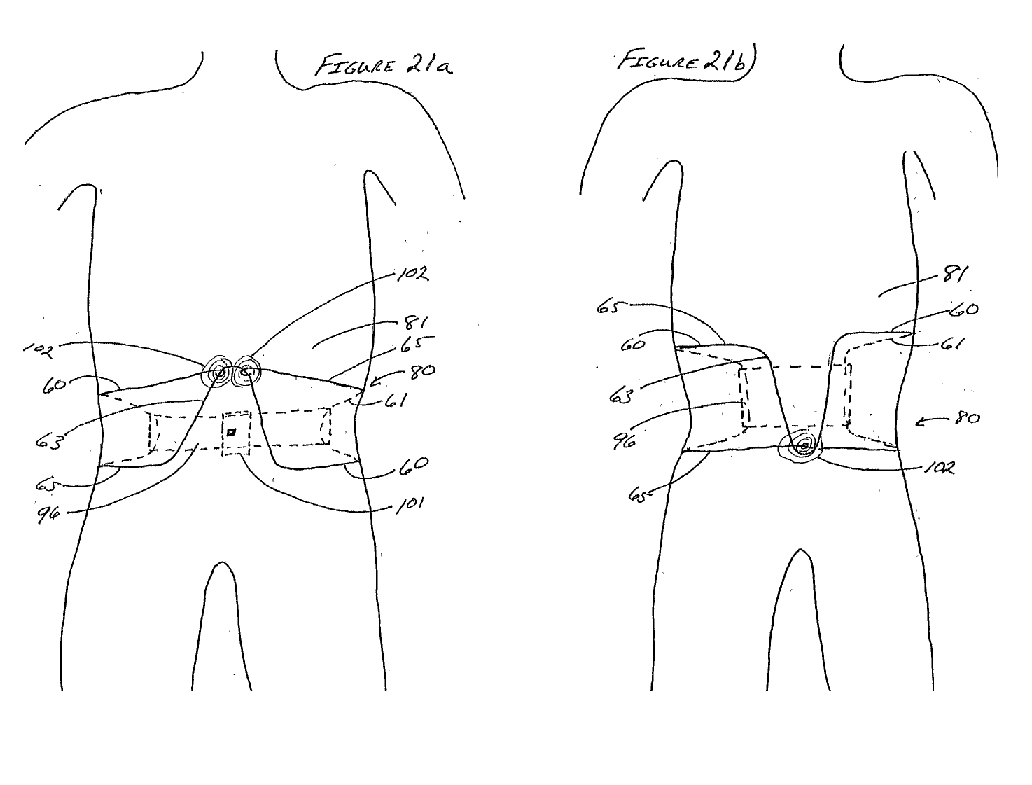

FIG. 23a shows an alternative back brace 80 embodiment fabricated with two superelastic wire supports (60) located on opposite sides of the spine. The back brace consists of a jacket fabricated from polyester, cotton, Kevlar, or other fabric or material and encompassing superelastic wire supports (60) embedded between layers of the jacket. Wire holders 132 maintain the position of adjacent superelastic wire supports (60).

FIG. 23b shows an alternative back brace 80 embodiment. This back brace 80 is fabricated from at least one superelastic wire support 60 preshaped in a jacket configuration. The at least one superelastic wire support 60 extends around the back to reinforce the back at multiple places. The superelastic wire support 61 also extends partially around the front of the body at multiple locations where attachment mechanisms 96 are used to tighten and secure the back brace to the body. Locking mechanisms 101 are used to maintain the attachment mechanisms 96 in their secured orientation and permit releasing of the attachment mechanisms for removal of the back brace. This back brace embodiment 80 reinforces the entire back from the lower back to the neck.

FIGS. 23c to e show an end view of the back brace 80 taken along line B-B. The back brace 80 incorporates superelastic wire supports 60 extending from the front of the body where they are secured to the attachment mechanisms 96 through the back of the body. Along the back, or alternatively any region of the brace, any section of the at least one superelastic wire support 60 may be preformed into any desired shape to accommodate changes in the anatomy, follow contours, or apply spring force against particular anatomic structures. For example, the shape of the superelastic wire support 60 in FIG. 23d is designed to fit within the groves adjacent the spine. This helps stabilize the spine and applies force against the muscles adjacent the spine to, among other benefits, massage the muscles. The superelastic wire support 60 geometry in FIG. 23e incorporates a loop to provide a cushioning against the spine or applies continuous spring force against the spine or adjacent structures. Other changes in the resting shape of the superelastic wire supports may be used to target other applications.

The ability to tailor the superelastic wire supports may apply to indications that require very specific reinforcement properties. For example, pregnant women require back braces that accommodate changes in the belly as the baby grows. In this case the superelastic wire supports must address expanding protrusions in the belly yet still reinforce the back. To accomplish this the tightening of the back brace, such as the embodiment in FIG. 22c, may decrease the degree of the protrusions for early periods in the pregnancy. By loosening the back brace tightening mechanism 130, the protrusions may extend further outward to continue to reinforce the back but allow space for the belly to fit.

Superelastic wire (or other geometry) supports are also applicable to traction devices for the back. The superelastic wire supports may be shaped and secured at discrete locations to vary and tailor the stiffness profile of the back traction device to the desired application. The benefits of superelastic wire supports in back traction devices over rigid components is that the superelastic wire supports provide decompression to reduce stresses exerted on the spine while reinforcing the spine and other structures of the back. The spring force induced by deflection of the superelastic wire supports facilitates correct posture by straightening the back after the traction device wearer bends down to pick up an object, bends at the waist to sit, or otherwise moves in a direction the wearer eventually and controllably desires to reverse. Other traction device designs are unable to apply a spring force in response to a deflection and are extremely expensive since they require several pneumatically controlled bladders that are filled with air or fluid, or pads that are tightened with elastic bands.

The superelastic supports provide decompression to the spine and other back structures by exerting extension or separation forces strategically throughout the back, thereby decreasing the stress and strain exerted on the back. This aids in healing by preventing constant aggravation of the injured tissue, and enables the patient to resume daily activities despite having limited range of motion and strength of the back muscles. The traction devices also prevent compression-related pain commonly associated with back injuries. Superelastic wire supports are also relatively inexpensive and accommodate the variability in stiffness required for back traction devices.

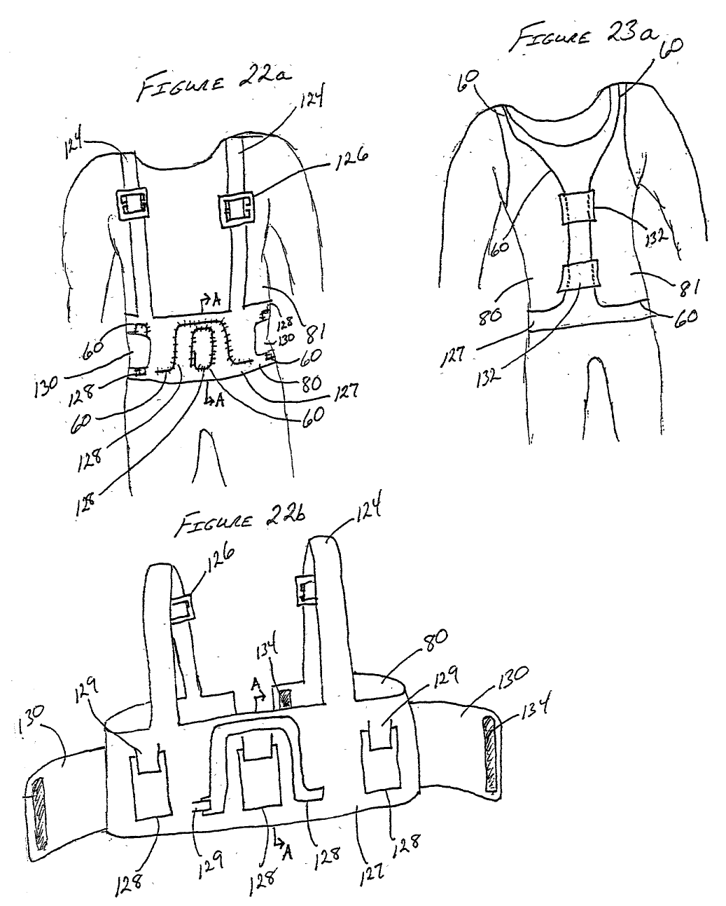

FIGS. 24a and b show an end view and a flattened view of a back traction device embodiment 80 that consists of a combination of tightening links 65 and spring links 63 designed to direct rotation and bending of the back relative to the hips and provide decompression of the spine and other structures of the back to aid in healing, and decrease the stress and strain exerted upon the back. These back traction devices reinforce the back muscles and spine to prevent spasms or injuries to the back, and enhance the movement of the back to induce an additional force during an activity. The spring links 63 of these back traction device embodiments induce a spring force designed to counteract gravity and decrease the stress on the back. The spring links also facilitate straightening the back from a bent orientation. The back traction device embodiments in FIGS. 24a and b consist of single superelastic wire supports 60 thermally formed into the pattern of tightening links and spring links shown. Of course, multiple superelastic wire supports 60 may alternatively be used to form the traction device structure. Interlocking mechanisms 102 (not shown) may be used to attach the free ends of the superelastic wire support 60. Two attachment mechanisms 96 are bonded to opposite sides of the traction device and incorporate a removable anchoring means that enables tightening, securing, and removing the back traction device. The anchoring means of the attachment mechanism may consist of Velcro, a ratcheting means, a buckle, a screw-type mechanism, or other temporary and adjustable securing device.

The two tightening links 65 are strategically positioned with one horizontal tightening link supported by the hips, and the second horizontal tightening link spaced apart from the first by at least one spring link and supporting the back at the ribcage or underarms. The tightening links are independently attached at the front of the traction device but remain integrated by the at least one spring link. The spring links may be integrated and designed to exert a consistent decompression force against the back or may be independently controlled or designed with varying spring characteristics and stiffness to alter the decompression forces exerted throughout the back. Providing decompression forces against the back reduces the stresses on the spine, adjacent back muscles, or other back anatomy. The spring links and tightening links of the traction devices may also be configured to apply radial compression forces to stabilize the position of the spine and back structures while simultaneously exerting axial decompression forces to reduce the stresses on the spine and back structures.

FIGS. 25a and b show an alternative back traction device embodiment incorporating at least one superelastic wire support 60. This back traction device 80 incorporates at least one superelastic wire support 60 formed in a looping pattern of spring links 63 with consistent or varying heights. The combined bases of each looping wire support form the lower tightening link 65. The apex of each looping wire support is attached to an upper tightening link 65 with interconnects 102. The degree of engagement for the tightening links 65 determines the height of the traction device, and the force exerted by the spring links 63 against the ribcage, the spine, or other structure designed to reduce the stress applied to the anatomy during rest, or daily activities.

Tautening the tightening links 65 adjusts the height of the spring links, changes the degree of radial reinforcement, and adjusts the decompression forces exerted against the back. Altering the height of the traction device may be used to accommodate variations in anatomy (for example, shorter heights may be required to position below the ribcage). The ability to tailor the height of the back brace also accommodates differences in desired surface reinforcement, spring characteristic, or stiffness. This traction device 80 embodiment incorporates at least one covering layer (not shown) encompassing the at least one superelastic wire support 60. The traction device 80 incorporates a first set of locking mechanisms 96 to attach the traction device at the front. Shoulder straps (not shown) may also be used to stabilize the back brace to the shoulders and shoulder strap locks may enable altering the shoulder straps to accommodate various patient anatomical shapes.

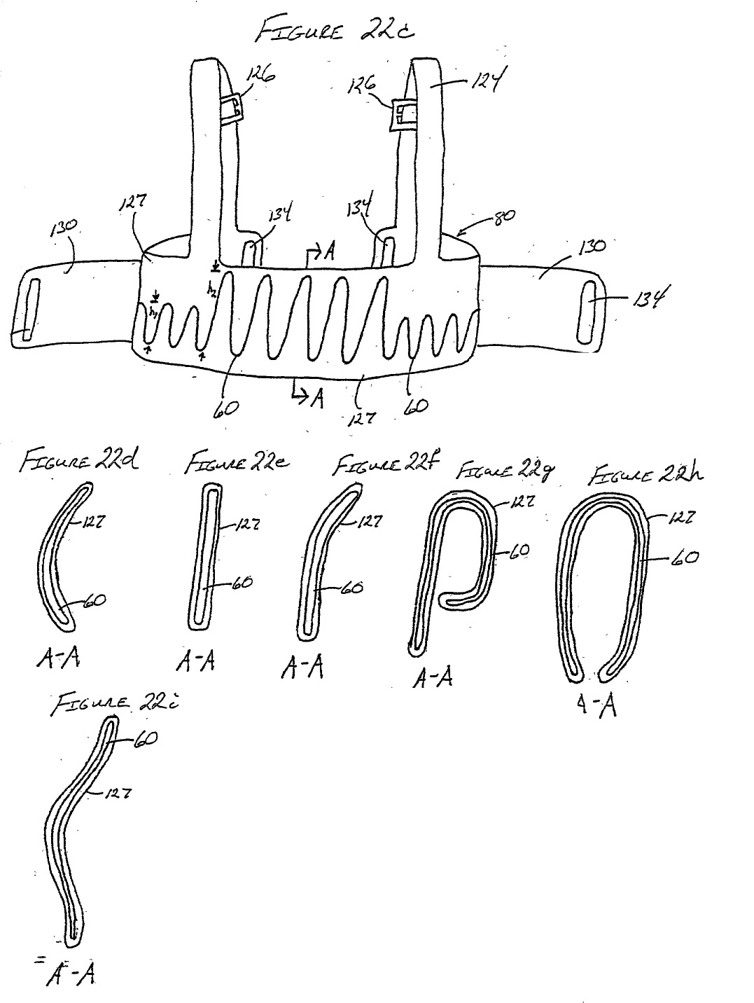

FIGS. 26a and b show an alternative back traction device 80 embodiment fabricated with superelastic wire supports (60) fabricated in looping sections connected at the apex of the looping sections to tightening links with interconnects 102. The looping sections define the spring links 63 and the characteristics of the spring links (height, coverage, spring constant, etc.) depends on the amount of deflection of the looping spring links by adjusting the tightening links 65. In addition, the back traction device incorporates stiffening straps 83 to further vary the degree of decompression applied to the back by altering the separation of the spring links 63 and the extension force exerted by the superelastic wire supports. As the stiffening straps 83 are tightened, opposing segments of the looping spring links 63 are brought together, thereby increasing the height of the spring links and the decompression force exerted by the spring links. As the stiffening straps 83 are loosened, opposing segments of the looping spring links 63 are allowed to separate towards their preformed configuration, thereby decreasing the height of the spring links and the decompression force exerted by the spring links. The stiffening straps 83 in this embodiment are shown as a single strap; however, multiple straps may alternatively be used to provide independent actuation of the spring links and provide more variability in stiffness throughout the back traction device.

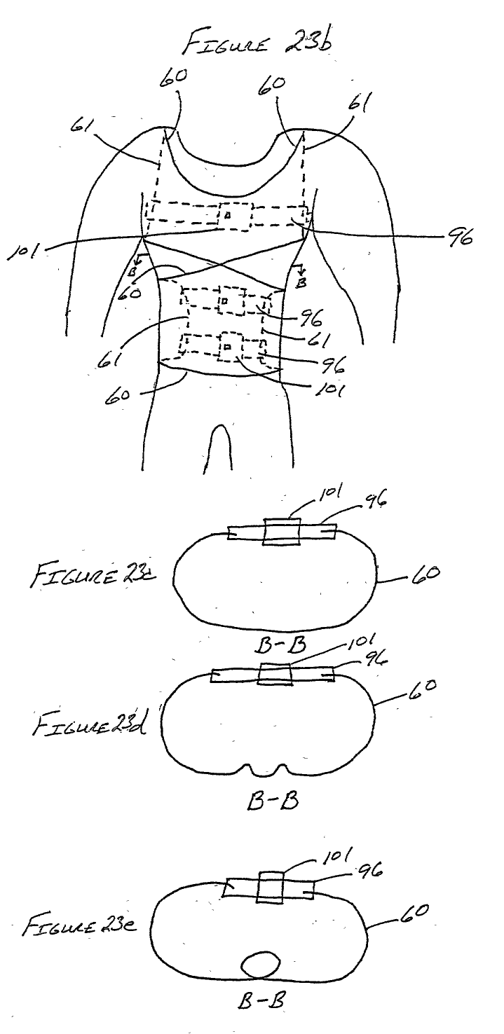

FIGS. 27a and b show an alternative back traction device 80 embodiment. This back traction device 80 is fabricated from at least one superelastic wire support 60 preshaped in a jacket configuration, extending from the waist to the underarm region of the back. The at least one superelastic wire support 60 extends around the back to reinforce the back at multiple places. The superelastic wire supports also extend partially around the front of the body at multiple locations where attachment mechanisms 96 are used to tighten and secure the back traction device to the body. The superelastic wire supports 60 in this embodiment are fabricated as adjacent loops located on top of each other. The adjacent loops are attached with interconnects 102 and the opposite ends are attached to tightening links 65 with interconnects 102. Stiffening straps 83 are attached to each side of the looping spring links 63 providing independent actuation of the spring links for increased flexibility in determining the amount of decompression the back traction device exerts on the back.

Along the back, or alternatively any region of the brace, any section of the at least one superelastic wire support 60 may be preformed into any desired shape to accommodate changes in the anatomy, follow contours, or apply spring force against particular anatomic structures. For example, the shape of the superelastic wire support 60 in FIG. 27a may be designed to fit within the groves adjacent the spine. This helps stabilize the spine and applies force against the muscles adjacent the spine to, among other benefits, massage the muscles. The superelastic wire support 60 geometry in FIG. 27a may alternatively incorporate a loop to provide a cushioning against the spine or applies continuous spring force against the spine or adjacent structures. Other changes in the resting shape of the superelastic wire supports may be used to target other applications.

The ability to tailor the superelastic wire supports may apply to indications that require very specific reinforcement properties. For example, pregnant women require back traction devices that accommodate changes in the belly as the baby grows. In this case the superelastic wire supports must address expanding protrusions in the belly yet still provide decompression forces and reinforce the back. To accomplish this the tautening of the back traction device, such as the embodiment in FIG. 27a, may decrease the degree of the protrusions for early periods in the pregnancy. By loosening the back brace tightening mechanism 130, the protrusions may extend further outward to continue to aid the back but allow space for the belly to fit.

A variety of congenital defects and injuries require spring forces applied to anatomic structures of the back. For example, compression and torsion injuries that damage the vertebral endplate or the intervertebral discs may be treated or prevented by forming the superelastic wire supports of the back brace to apply decompression and stabilizing forces to the vertebrate and surrounding anatomy. Congenital defects of the spine may be treated by continuously applying forces (decompression, tensile, or compression) against the spine to slowly reshape or stabilize the spine.

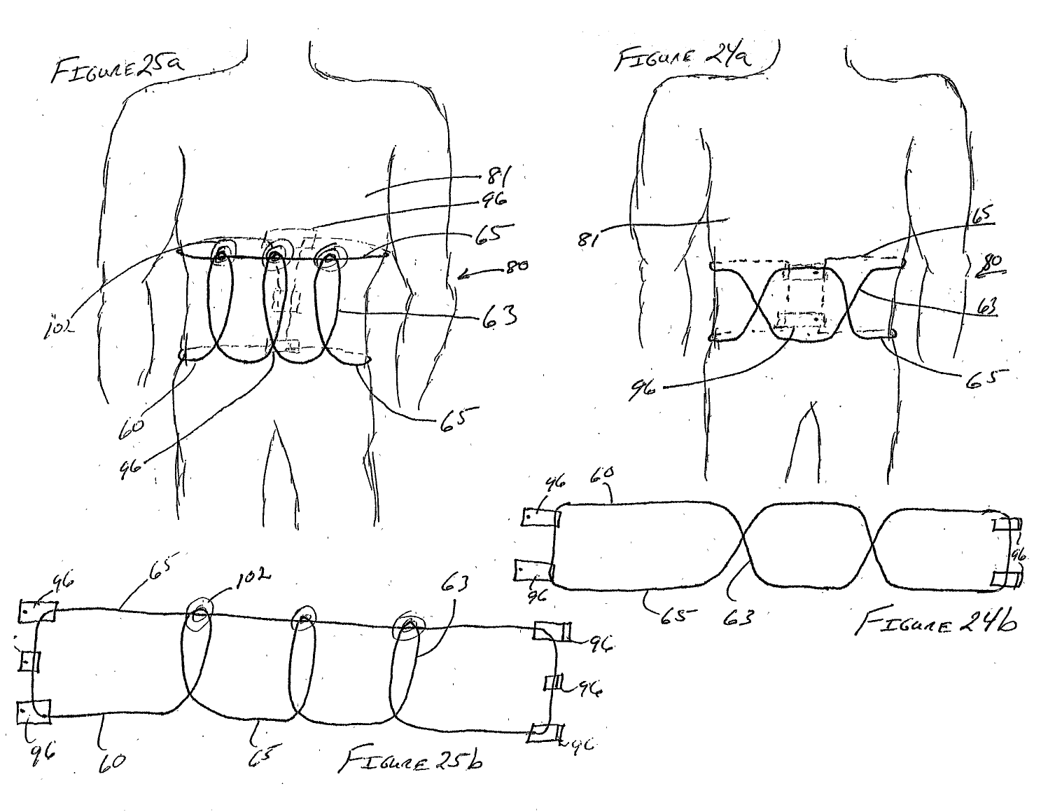

The superelastic supports incorporated in back traction devices (or other anatomic braces) may be fabricated from a flat sheet cut into the desired pattern of tightening links 65 and spring links 63. Alternatively, as shown in FIGS. 28a and b, the superelastic supports may be fabricated with a combination of flat sheet material cut into the desired pattern and wires attached to regions of the flat sheet material.

The properties of the superelastic supports or structures described above may be varied to address applications in which the stiffness or elasticity needs to be varied accordingly. The composition of the superelastic material may be chosen to select the temperature range in which the support members or structures exhibit stress-induced martensite. As such, the amount of austenite, and stress-induced martensite characteristics throughout a specific temperature range may be chosen to specify the degree of deflection and amount of force exerted by the superelastic support member once deflected. For example, the superelastic properties of the material may be chosen so as exercise (or other activity) increases, the associated temperature increase induces a change in the superelastic properties of the superelastic support member or structure to provide, for example, increased rigidity and/or elasticity of the material.

The various traction devices, braces and scaffolds described above may incorporate additional components such as magnets, padding, cold packs or drug-eluting coverings to further enhance the performance of the brace and scaffolds for their intended purposes.

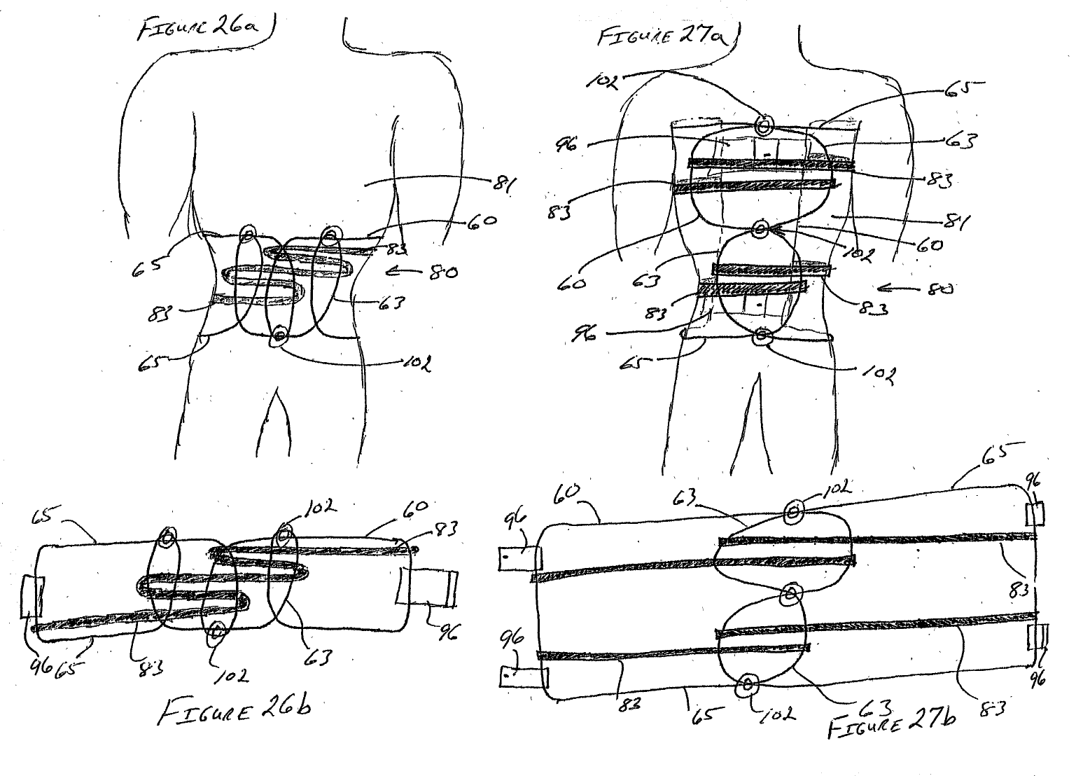

FIGS. 29a and b show neck braces 94 fabricated with superelastic supports 60. The neck brace 94 in FIG. 29a incorporates a rib and backbone configuration with a vertical backbone 98 containing ribs 100 that extend around the neck 95. Ends of the ribs 100 may be tied or otherwise anchored together using knotted strings, Velcro, or other removable attachment means 96. The neck brace 94 in FIG. 29b consists of a first tightening link 65, a second tightening link 65, and two spring links 63. The tightening links 65 are locked together with knotted strings, Velcro, or other removable attachment means 96. The neck brace restricts motion of the head relative the shoulder to prevent whiplash, or other injury that results from abrupt or excessive movement of the neck.

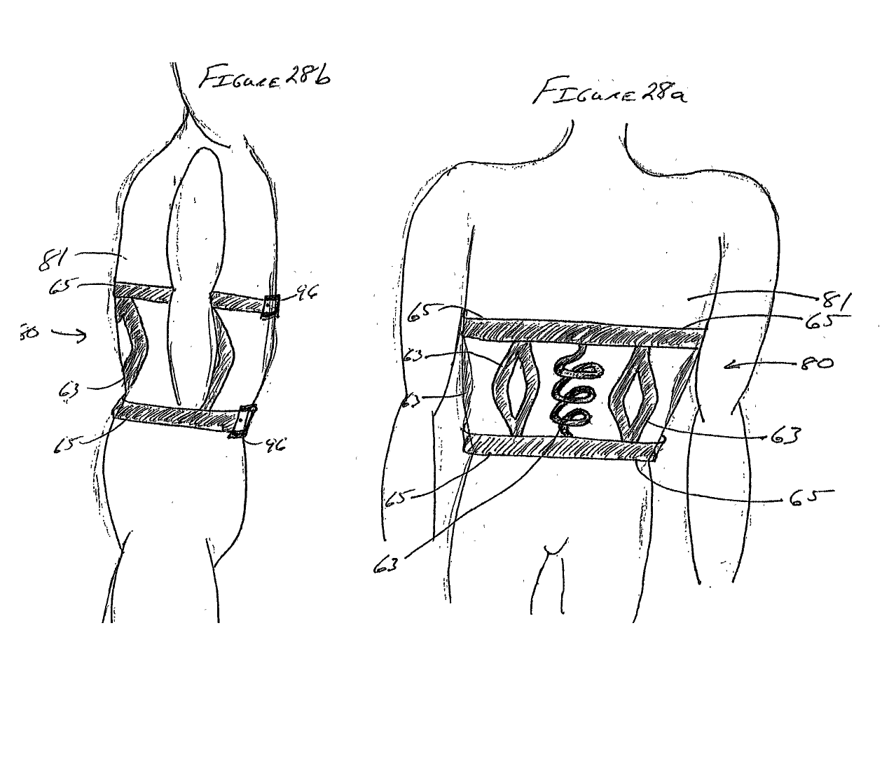

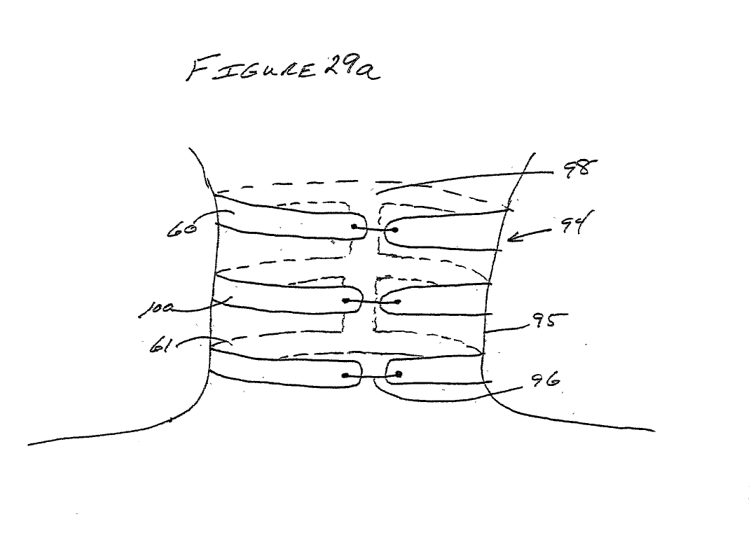

FIGS. 30 and 31 show neck braces 94 fabricated with superelastic supports 60. The neck brace 94 embodiment in FIG. 30 incorporates a superelastic wire support 60 extending from the chest, around the underarm, along the back 81 and neck 95, to a helmet, hat, or other head restraint 156, back along the neck 95 and back 81, around the opposite underarm 162, and back to the chest 164. Crossing segments of the superelastic wire support 60 located along the back are attached with an interlocking mechanism 102 just below the neck. Opposite sides of the back brace located at the chest 164 are removably secured using an attachment mechanism 96.

The neck brace 94 embodiment in FIG. 31 incorporates two superelastic wire supports 60, each extending from the chest 164 around an underarm 162, along the back 81, to one side of a helmet, hat, or other head restraint 156, along the side of the head restraint, and back to the chest 164. Adjacent sections of the superelastic wire supports 60 located along the back 81 are bonded using at least one interlocking mechanism 102. Opposite sides of the back brace located at along the chest 164 region may be removable secured using an attachment mechanism 96.

The neck brace embodiments 94 shown in FIGS. 30 and 31 permit a predetermined degree of rotation about the neck but prevents abrupt or excessive forward, backward, or sideways bending at the neck; such dramatic, sudden motions are commonly associated with injury to the neck during a car accident, an impact against the head, or other event capable of damaging the vertebrate, spine, neck muscles, or other upper back or neck anatomy.

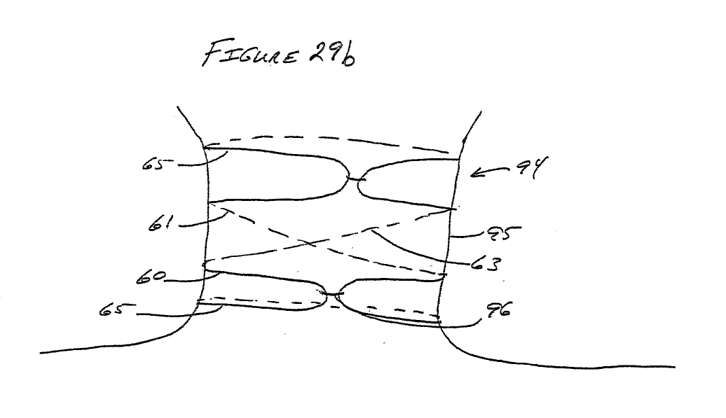

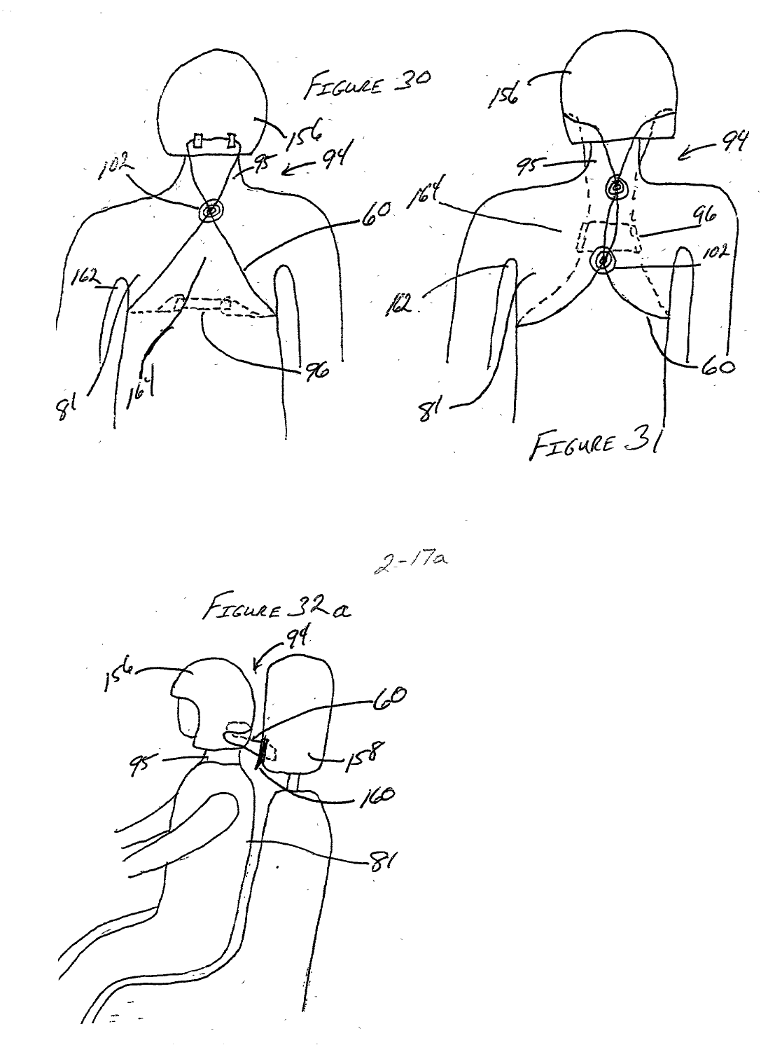

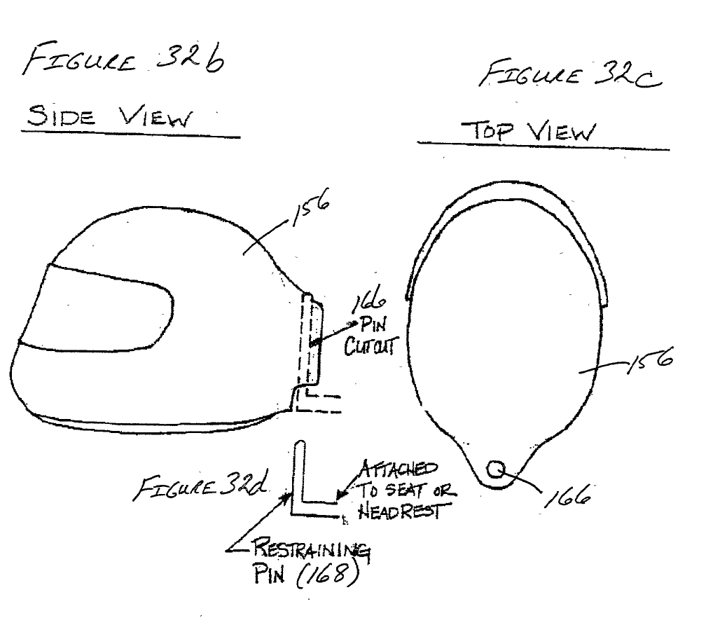

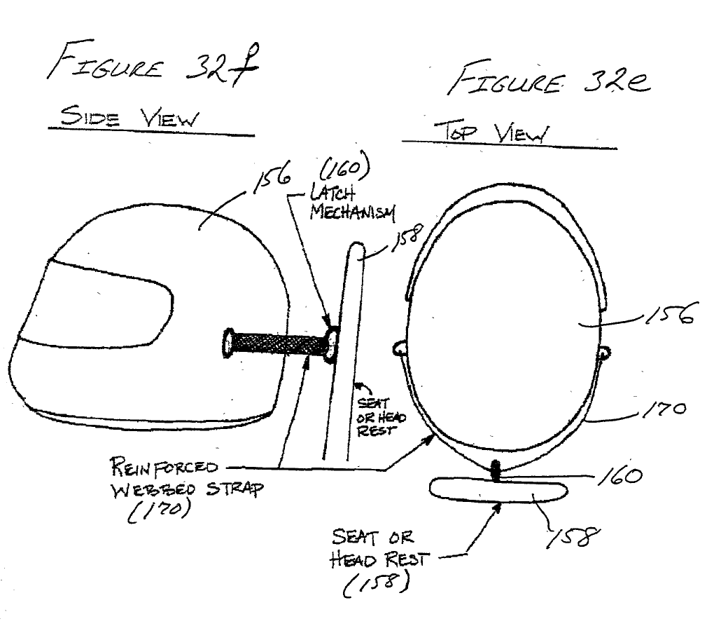

FIG. 32a shows an alternative neck brace that removably secures a helmet or other head restraint 156 to seat of a car, or a separate collar brace (not shown). The neck brace consists of a superelastic wire support 60 extending from the car seat 158 or collar brace to the helmet or other head restraint 156, along the helmet, and back to the car seat. The superelastic wire support 60 permits a predetermined degree of rotation, and prevents abrupt or excessive forward, backward, or sideways bending at the neck by removably securing the helmet or other head restraint to a car seat or other anchor. This neck brace embodiment incorporates a rapid release mechanism 160 to ensure the helmet and head restraint wearer can quickly and deliberately separate from the car seat or other anchor.

FIGS. 32b and c show an alternative head restraint embodiment. This head restraint 156 incorporates a pin cutout 166, which allows a restraining pin 168, shown in FIG. 32d, to pass and be attached. The other end of the restraining pin 168 is attached to the headrest 158 or other component of a car, other motorized vehicle, or other stabilization component. The restraining pin 168 incorporates teeth, threads, or other locking component that matches the removable attachment mechanism in the cutout region 166 of the head restraint 156. A safety release (reachable by the wearer of the head restrain) disengages the restraining pin 168 from the track 166 of the head restraint allowing the restraining pin to be removed from the track 166. The restraining pin 168 allows unobstructed, horizontal movement of the head restraint 156, yet prevents excessive movement relative to the constrained torso of the head restraint wearer during a frontal impact crash or other dramatic change in motion.

FIGS. 32e and f show an alternative head restraint embodiment. This head restraint 156 incorporates reinforced straps 170 permanently attached to the head restraint 156 at two or more locations, and passing through a latch mechanism 160, which is bonded to the headrest 158 or other component. The latch mechanism incorporates a hole, slot, or other feature for the reinforced straps 170 to pass and permit limited rotation of the head restraint 156. The reinforced straps themselves may be fabricated from superelastic wires interwoven with conventional materials to increase the tensile strength and abrasion resistance of the straps. Alternatively, superelastic wire, rod, band, sheet, or other geometry may be bonded to the top, and/or bottom of the straps to provide significant abrasion resistance. The same may be true of other straps including seat belts, window nets, ropes, etc.

The superelastic wire support structures described above may be used in braces that reinforce, guard, or shield the ribs, forearm, skull, thigh, hip, shin, other bone, other joint, or other anatomic structure to prevent localized stressing that may result in fracture, bruising, tearing, or other injury. Alternatively, these support member structures may be incorporated in braces intended to reinforce sprained joints, fractured bones, or other injuries in which the joint or anatomic region must be reinforced or stabilized so they heal properly. The ability to thermally shape the support member structures to any form enables customizing the support member structures to the patient's anatomy. In addition, these support member structures may be used in braces that reinforce the sternum or ribs and apply continuous force against the sternum, ribs, or other wound after open chest surgery involving a medium sternotomy, thoracotomy, or thoracostomy, or other invasive procedure. This facilitates and quickens healing of the sternum, ribs, or other anatomy after surgery.

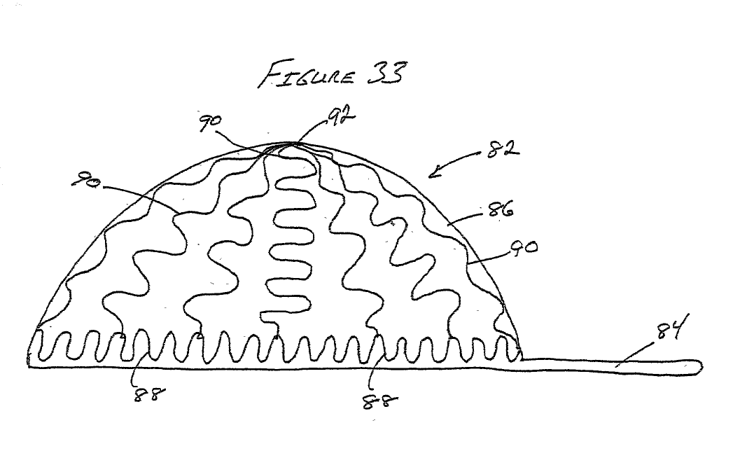

The superelastic support may be used to prevent localized over-stressing, which may result in fracture of bones or other tissues. FIG. 33 shows a hat 82 containing superelastic supports configured to distribute localized stressing over a large surface. The embodiment in FIG. 33 shows a single layer of superelastic supports; alternatively two or move layers of superelastic supports may be connected and spaced to better distribute localized forces over a greater surface area. Such superelastic support structures may be utilized for any bone or tissue region that has the potential to be exposed to localized over-stressing.

Superelastic supports 60 (wire or other raw material geometry) may also be used in implantable or external bone supports that secure bone fractures during healing, promote bone growth, or otherwise stabilize the position of adjacent bone segments at a joint. The superelastic wire supports 60 enable incorporating a hinge mechanism in the bone support without the need for a pivoting mechanism between adjacent sections of the support. As such the bone support may be fabricated from an integrated component fabricated to elicit the desired stiffness profile, spring characteristic, and flexion capabilities.

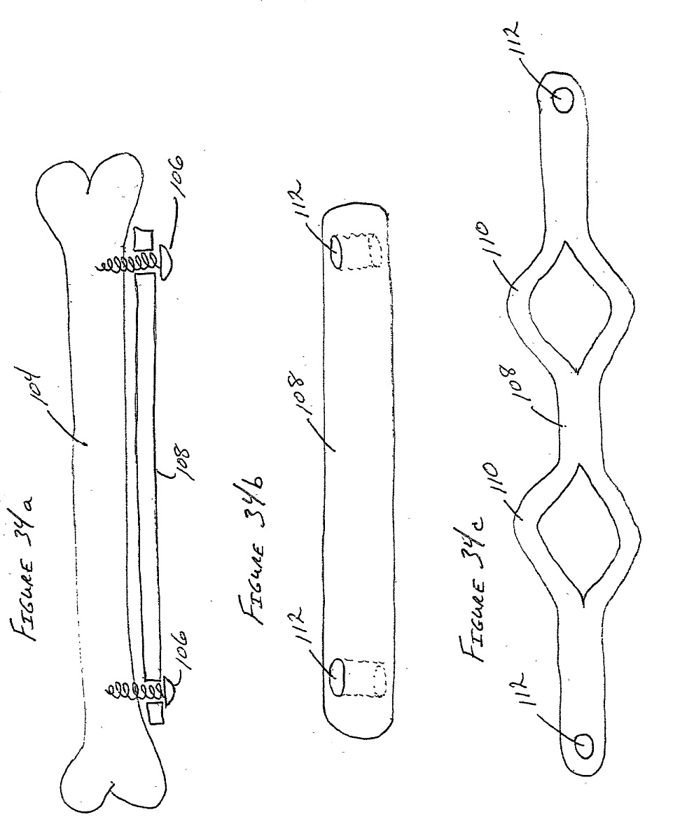

FIG. 34a shows a bone 104 that is reinforced with a bone support 108 fabricated from a superelastic material. The bone support 108 consists of a rod, band, or ribbon of superelastic material having holes 112 at predetermined attachment locations along the bone support, as shown in FIG. 34b. Bone screws 106 are inserted through the bone support holes 112 and into the bone 104 at desired locations along the bone support, as shown in FIG. 34a. This bone support embodiment secures bone fragments and ensures close apposition of such fragments to encourage healing of the bone. The bone support 108 may reside exterior to the skin with only the bone screws 106 implanted in the body, or may be implanted along the bone for permanent reinforcement of the bone.

FIG. 34c shows a bone support 108 that incorporates two support springs 110 along the bone support. The support springs 110 may be fabricated by laser cutting or otherwise forming a longitudinal channel through the bone support 108 and thermally forming the sides of the channel in an open orientation, as shown in FIG. 34c. The open channels produce springs that urge the bone support to lengthen when compressed longitudinally. This produces a force designed to provide continued longitudinal force capable of promoting bone growth. The bone support also incorporates holes 112 at predetermined locations along the bone support for fixation to the bone(s).

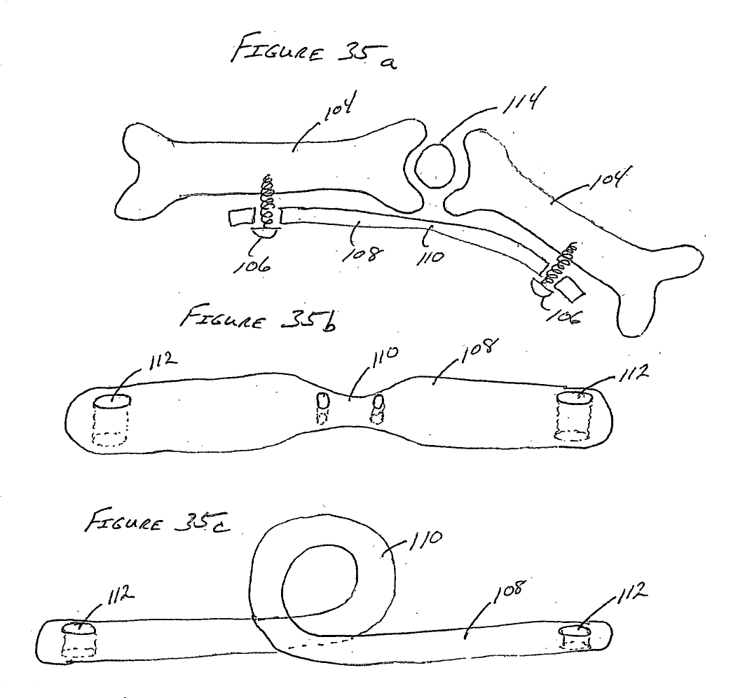

FIG. 35a shows a bone support 108 that incorporates a support spring 110 that function as a hinge. This bone support is attached to adjacent bones 104 about a joint 114 and maintains the position of the adjacent bones yet allows directed rotation of the bones about the joint. The bone support embodiment shown in FIG. 35b incorporates a reduced width (or diameter) section that produces the support spring 110 and functions as the hinge. The bone support 108 incorporates holes 112 through which bone screws 106 attach the bone support 108 to the adjacent bones. The bone support embodiment in FIG. 35b also incorporates slots, notches, or other features that further define the characteristics (e.g. the spring constant, hinge location, etc.) of the support spring. The bone support embodiment shown in FIG. 35c incorporates a single loop that defines the support spring 110. As previously stated, the bone supports may be located external to the skin with only the bone screws penetrating into the body, or implanted in proximity to the bones.

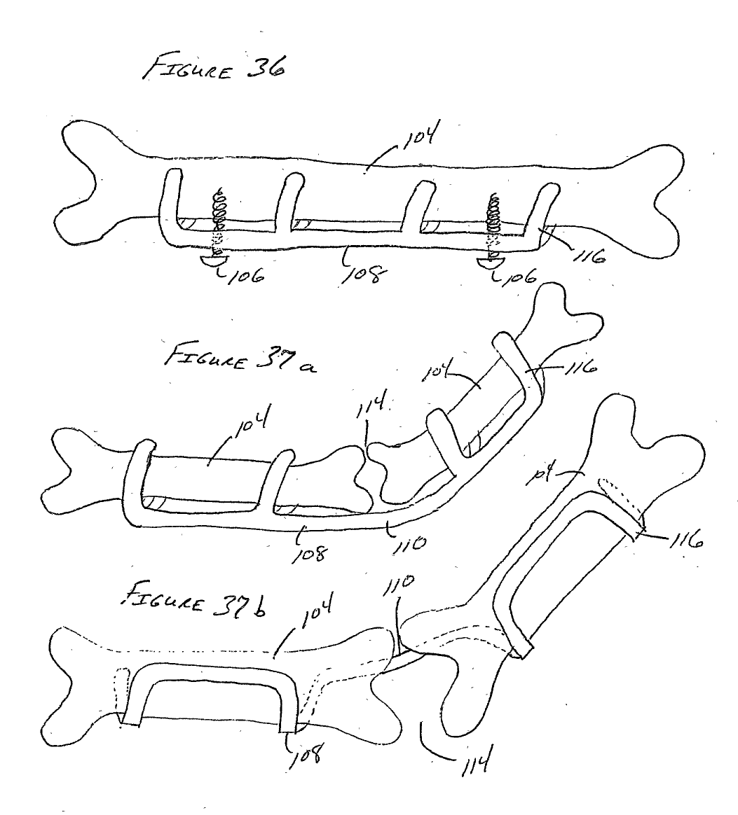

FIG. 36 shows another bone support embodiment designed to stabilize fractured bones. This bone support 108 further incorporates radial wings 116 that extend partially around the bone to further stabilize the bone. The bone support 108 may also incorporate holes 112 that bone screws 106 may pass and secure the bone support to the bone segments at desired locations. This bone support embodiment may be implanted such that the wings 116 extend partially around and contact the bone or may be located external to the skin such that the wings extend partially around the arm, leg, or other body part encompassing the fractured bone.

FIGS. 37a and b show two bone support embodiments that incorporate at least one spring link 110 to define the at least one hinge that permits bending of the bones about a joint 114. The bone supports 108 incorporate at least one wing 116 to further stabilize the bone support relative to the bones. Again, the bone support may be implanted such that the wings extend partially around and contacts the bones, or located external to the skin with the wings extending partially around the arm region, leg region, or other body parts in proximity to the joint.

Although the present inventions have been described in terms of the preferred embodiments above, numerous modifications and/or additions to the above-described preferred embodiments would be readily apparent to one skilled in the art. It is intended that the scope of the present inventions extend to all such modifications and/or additions and that the scope of the present inventions is limited solely by the claims of the invention.

We claim as our invention:

1. An orthopedic device for reinforcing an anatomic joint of a body comprising:
at least one superelastic member;
a covering encapsulating said superelastic member;
wherein said superelastic member comprises at least one tightening link that secures the orthopedic device to the body, and at least one spring link comprising an integrated flex region that defines the motion of the anatomic joint;

2. The device of claim 1 wherein said spring link comprises at least one loop at the flex region that applies a first resistance when deflected in a first direction and a second resistance, different from the first, when deflected in a second direction;

3. The device of claim 1 further comprising a stop associated with said spring link that limits deflection of the flex region in a first direction and preserves deflection in a second direction different from the first direction;

4. The device of claim 1 wherein the device comprises two tightening links interconnected with said spring link such that the spring link limits the angular rotation of said tightening links relative to each other;

5. The device of claim 1 wherein said tightening link is deflected, with an external force, into an enlarged configuration for placement and returns towards its resting configuration once the external force is removed;

6. The device of claim 1 wherein said tightening link incorporates a locking mechanism to removably secure the tightening link to the body,

7. An orthopedic device for limiting the motion of an anatomic joint of a body comprising:
at least one superelastic member comprising at least two tightening links, one tightening link located on each side of the anatomic joint, and at least two spring links connecting the tightening links;
wherein each tightening link and each spring link is adapted to deflect from a first configuration to a second configuration different from said first configuration in response to an external force, and return towards said first configuration upon reduction or removal of said external force;

8. The device of claim 7 further comprising at least one interconnect mechanism securing intersecting regions of said spring links;

9. The device of claim 7 further comprising at least one locking mechanism associated with at least one of said tightening links;
wherein said locking mechanism removably secures said tightening link to said body;

10. The device of claim 7 wherein at least one of said spring links further comprises a stop mechanism adapted to limit motion of the joint in a first direction and preserve motion of the joint in a second direction different from the first direction;

11. The device of claim 7 wherein at least one of said spring links is adapted to provide resistance to motion of the anatomy about the joint;

12. An orthopedic device for applying decompression to an anatomic structure of a body comprising:
at least one superelastic member comprising at least two tightening links, and at least one spring link connecting the tightening links and adapted to apply an outward force against the tightening links;
at least one locking mechanism adapted to secure the tightening links to the anatomic structure; and
at least one decompression mechanism associated with said spring link to define the magnitude and the direction of the outward force exerted by said spring link;

13. The device of claim 12 wherein said tightening links are integrated with said spring links;

14. The device of claim 12 wherein said spring links are adapted to also apply a directional force against the anatomic structure;

15. The device of claim 12 wherein a plurality of spring links connect said tightening links, and said decompression mechanism is adapted to adjust the lateral separation of said spring links to alter said outward force;

16. The device of claim 12 wherein said decompression mechanism is adapted to adjust the vertical separation of said tightening links to alter said outward force;

17. The device of claim 12 wherein said spring link is adapted to deflect along a second plane, different from said decompression plane, in response to an external force, and return towards its resting configuration once the external force is reduced or removed;

18. The device of claim 12, wherein said at least one superelastic member comprises nickel titanium;

19. The device of claim 12, wherein said at least one superelastic member comprises a single rod of material thermally formed into said tightening links and the at least one spring link;
